(19) United States
(12) Patent Application Publication
(10) Pub. No.: US 2003/0152942 A1
(43) Pub. Date: Aug. 14, 2003

(54) Nucleic acid detection in pooled samples

(76) Inventors: Lance Fors, Madison, WI (US); Bruce P. Neri, Madison, WI (US); Mary Ann D. Brow, Madison, WI (US); Monika de Arruda Indig, Madison, WI (US); Robert Roeven, Stoughton, WI (US)

Correspondence Address: Jason R. Bond, MEDLEN & CARROLL, LLP, Suite 350, 101 Howard Street, San Francisco, CA, 94105, US

(21) Appl. No.: 10/142,283

(22) Filed: May 9, 2002

Related U.S. Application Data
(60) Provisional application No. 60/326,549, filed on Oct. 2, 2001.
(60) Provisional application No. 60/289,764, filed on May 9, 2001.

(51) Int. Cl. C12Q 1/70; C12Q 1/68

(52) U.S. Cl. 435/6; 435/5

(57) ABSTRACT

The present invention relates to detecting target nucleic acid sequences in pooled samples. In particular, the present invention relates to compositions and methods for detecting the presence or absence of target nucleic acid sequences (e.g. RNA virus sequences) in a pooled sample employing an INVADER detection assay. In certain embodiments, the present invention allows target nucleic acid sequence detection in pooled biological samples (e.g. pooled blood samples) without prior amplification of the target.

The present application claim priority to U.S. Provisional Application Serial No. 06/326,549, filed Oct. 2, 2001, and U.S. Provisional Application Serial No. 06/289,764, filed May 9, 2001, both of which are hereby incorporated by reference.

### FIELD OF THE INVENTION

The present invention relates to detecting target nucleic acid sequences in pooled samples. In particular, the present invention relates to compositions and methods for detecting the presence or absence of target nucleic acid sequences (e.g. RNA virus sequences) in a pooled sample employing an INVADER detection assay. In certain embodiments, the present invention allows target nucleic acid sequence detection in pooled biological samples (e.g. pooled blood samples) without prior amplification of the target.

### BACKGROUND OF THE INVENTION

Blood, plasma, and biological fluid donation programs are essential first steps in the manufacture of pharmaceutical and blood products that improve the quality of life and that are used to save lives in a variety of traumatic situations. Such products are used for the treatment of immunologic disorders, for the treatment of hemophilia, and are also used in maintaining and restoring blood volume in surgical procedures and other treatment protocols. The therapeutic uses of blood, plasma, and biological fluids require that donations of these materials be as free as possible from viral contamination. Typically, a serology test sample from each individual blood, plasma, or other fluid donation is tested for various antibodies, which are elicited in response to specific viruses, such as hepatitis C (HCV) and two forms of the human immunodeficiency virus (HIV-1 and HIV-2). In addition, the serology test sample may be tested for antigens designated for specific viruses such as hepatitis B (HBV), as well as antibodies elicited in response to such viruses. If the sample is serology positive for the presence of either specific antibodies or antigens, the donation is excluded from further use.

Whereas an antigen test for certain viruses, such as hepatitis B, is thought to be closely correlated with infectivity, antibody tests are not. It has long been known that a blood plasma donor may, in fact, be infected with a virus while testing serology negative for antibodies related to that virus. For example, a window exists between the time that a donor may become infected with a virus and the appearance of antibodies, elicited in response to that virus, in the donor's system. The time period between the first occurrence of a virus in the blood and the presence of detectable antibodies elicited in response to that virus is known as the “window period.” In the case of HIV, the average window period is approximately 22 days, while for HCV, the average window period has been estimated at approximately 70-98 days. Therefore, tests directed to the detection of antibodies, may give a false indication for an infected donor if performed during the window period, i.e., the period between viral infection and the production of antibodies. Moreover, even though conventional testing for HBV includes tests for both antibodies and antigens, testing by more sensitive methods have confirmed the presence of the HBV virus in samples which were negative in the HBV antigen test.

One method of testing donations, which have passed available antibody and antigen tests, in order to further ensure their freedom from incipient viral contamination, involves testing the donations by a polymerase chain reaction (PCR) method. PCR is a method for detecting the presence of specific DNA or RNA sequences related to a virus of interest in a biological material by amplifying the viral genome. Because the PCR test is directed to detecting the presence of an essential component of the virus itself, its presence in a donor may be found almost immediately after infection. There is, theoretically therefore, no window period during which a test may give a false indication of freedom of infectivity. A suitable description of the methodology and practical application of PCR testing is contained in U.S. Pat. No. 5,176,995, the disclosure of which is expressly incorporated herein by reference.

PCR testing is, however, very expensive and since the general donor population includes a relatively small number of positive donors, individual testing of each donation is not cost effective or economically feasible. Therefore, an efficient and cost-effective method of testing large numbers of blood or plasma donations to eliminate units having pathogenic (e.g. viral) contamination is needed.

### SUMMARY OF THE INVENTION

The present invention relates to detecting target nucleic acid sequences in pooled samples. In particular, the present invention provides compositions and methods for detecting the presence or absence of target nucleic acid sequences (e.g. RNA virus sequences) in a pooled sample employing an INVADER detection assay. In certain embodiments, the present invention allows target nucleic acid sequence detection in pooled biological samples (e.g. pooled blood samples) without prior amplification of the target.

In some embodiments, the present invention provides methods for performing nucleic acid testing on a pooled sample, comprising: a) providing; i) a pooled sample, wherein the pooled sample comprises biological material (e.g. biological fluid) combined from a plurality of individual samples; and ii) INVADER assay reagents configured to detect the presence or absence of a target nucleic acid sequence; and b) contacting the pooled sample with the INVADER assay reagents under conditions such that the presence or absence of the target nucleic acid sequence in the pooled sample is determined. In certain embodiments, the method does not require prior amplification of the target nucleic acid sequence.

In other embodiments, the present invention provides methods for performing nucleic acid testing on a pooled sample, comprising: a) providing; i) a plurality of individual biological fluid samples; and ii) INVADER assay reagents configured to detect the presence or absence of a target nucleic acid sequence; and b) forming a sub-pool by combining a portion of each of the plurality of individual biological samples, and c) contacting the sub-pool with the INVADER assay reagents under conditions such that the presence or absence of the target nucleic acid sequence in the sub-pool is determined. In some embodiments, the method does not require prior amplification of the target nucleic acid sequence prior to detection. In particular embodiments, the contacting indicates that the target nucleic acid sequence is absent from the sub-pool, and the method further comprises the step of combining the plurality of individual biological samples into a primary pool. In other embodiments, the contacting indicates that the target nucleic acid sequence is present in the sub-pool, and the method further comprises he step of screening each of the individual biological samples for the presence or absence of the target nucleic acid sequence. In certain embodiments, the biological fluid comprises blood (e.g. a blood donation from an individual). In other embodiments, the biological fluid comprises blood plasma.

In some embodiments, the target nucleic acid sequence is RNA. In other embodiments, the target nucleic acid sequence is DNA. In yet other embodiments, the target nucleic acid sequence is from a microorganism (e.g. a pathogenic microorganism). In preferred embodiments, the target nucleic acid sequence is from a virus. In some embodiments, the target nucleic acid sequences is from a pathogen selected from HIV-1, HIV-2, HCV, HBV, HTLVI, HTLV2, and HCMV.

In additional embodiments, the target nucleic acid comprises a first and second non-contiguous single-stranded regions separated by an intervening region comprising a double stranded regions, and wherein the INVADER detection reagents comprise; i) a bridging oligonucleotide capable of binding to the first and second non-contiguous single-stranded regions; ii) a second oligonucleotide capable of binding to a portion of the first non-contiguous single-stranded region; and iii) a cleavage means. In some embodiments, the contacting causes either the second oligonucleotide or the bridging oligonucleotide to be cleaved.

In particular embodiments, the plurality of individual samples are from a plurality of different individuals. In some embodiments, the plurality of individual samples comprises at least 5 individual samples. In other embodiments, the plurality of individual samples comprises at least 16 individual samples (e.g. 16, 20, 22, etc.). In some embodiments, the plurality of individual samples comprises at least 24 individual samples (e.g. 24, 30, 50, etc.). In other embodiments, the plurality of individual samples comprises at least 96 individual samples (e.g. 100, 200, 400, 500, or 1000). In some embodiments, the number is pre-determined using statistical modeling based on the expected prevalence of the target nucleic acid sequence.

In other embodiments, the method further comprises, prior to step b), the step of performing polymerase chain reaction (or other amplification method) on the pooled sample such that the target nucleic acid sequence is amplified if present in the pooled sample. In particular embodiments, the contacting step is performed under conditions such that the target nucleic acid sequence is not amplified before the presence or the absence of the target nucleic acid sequence is determined.

In some embodiments, the present invention provides methods for performing nucleic acid testing on a pooled sample, comprising: a) providing; i) a pooled sample, wherein said pooled sample comprises biological material combined from a plurality of individual samples; and ii) INVADER assay reagents configured to detect measure the quantity of a target nucleic acid sequence present in a sample; and b) contacting the pooled sample with the INVADER assay reagents under conditions such that the quantity of the target nucleic acid sequence present in said pooled sample is determined. In particular embodiments, the biological fluid comprises blood. In other embodiments, the biological material comprises blood plasma.

In some embodiments, the present invention provides methods for detecting target nucleic acid sequences in a pooled biological sample (e.g. blood sample) without prior amplification of the target nucleic acid sample. In particular embodiments, the number of individual samples in the pooled sample is at least 16 or at least 24.

In some embodiments, the target nucleic acid sequence to be detected is selected from HIV, HCV, HBV, Cytomegalovirus (CMV), human herpes virus 8 (HHV 8), Parvo B 19, HAV, and human t-cell leukemia virus (HTLV) I/II. In other embodiments, the target nucleic acid sequence to be detected is derived from a virus which virus includes, but is not limited to, Parvoviridae, Papovaviridae, Adenoviridae, Hepadnaviridae, Herpesviridae, Iridoviridae, and Poxviridae.

In further embodiments of the method the target nucleic acid is DNA, while in some preferred embodiments, the DNA is viral DNA. In yet other preferred embodiments, the virus includes, but is not limited to, Parvoviridae, Papovaviridae, Adenoviridae, Hepadnaviridae, Herpesviridae, Iridoviridae, and Poxviridae. For example, it is intended that the present invention encompass methods for the detection of any DNA-containing virus, including, but not limited to parvoviruses, dependoviruses, papillomaviruses, polyomaviruses, mastadenoviruses, aviadenoviruses, hepadnaviruses, simplexviruses [such as herpes simplex virus 1 and 2], varicelloviruses, cytomegaloviruses, muromegaloviruses, lymphocryptoviruses, thetalymphocryptoyiruses, rhadinoviruses, iridoviruses, ranaviruses, pisciniviruses, orthopoxviruses, parapoxviruses, avipoxviruses, capripoxviruses, leporipoxviruses, suipoxviruses, yatapoxviruses, and mulluscipoxvirus). Thus, it is not intended that the present invention be limited to any DNA virus family. In further embodiments of the method the target nucleic acid is RNA, while in some preferred embodiments, the RNA is viral RNA. In yet other preferred embodiments, the virus is selected from the group of Pieornaviridae, Caliciviridae, Reoviridae, Togaviridae, Flaviviridae, Orthomyxoviridae, Paramyxoviridae, Arenaviridae, Rhabdoviridae, Coronaviridae, Bunyaviridae, and Retroviridae. For example, it is intended that the present invention encompass methods for the detection of RNA-containing virus, including, but not limited to enteroviruses (e.g., polioviruses, Coxsackieviruses, echoviruses, enteroviruses, hepatitis A virus, encephalomyocarditis virus, mengovirus, rhinoviruses, and aphthoviruses), caliciviruses, reoviruses, orbiviruses, rotaviruses, birnaviruses, alphaviruses, rubiviruses, pestiviruses, flaviviruses (e.g., hepatitis C virus, yellow fever viruses, dengue, Japanese, Murray Valley, and St. Louis encephalitis viruses, West Nile fever virus, Kyanasur Forest disease virus, Omsk hemorrhagic fever virus, European and Far Eastern tick-borne encephalitis viruses, and louping ill virus), influenzaviruses (e.g, types A, B, and C), paramiyxoviruses, morbilliviruses, pneumoviruses, veisculoviruses, lyssaviruses, filoviruses, coronaviruses, bunyaviruses, phleboviruses, nairoviruses, uukuviruses, hantaviruses, sarcoma and leukemia viruses, oncoviruses, HTLV, spumaviruses, lentiviruses, and arenaviruses).

As mentioned above, the present invention provides methods of testing pooled biological fluid samples (e.g. using the INVADER assay). One reason to test biological fluid samples (such as blood or blood plasma) is to prevent the spread of infection diseases. Examples of infections diseases that may be tested for include, but are not limited to, Acanthamoeba—(Parasitical); Actinobacillus—Actinomycetemcomitans (Bacterial); Acute hemorrhagic conjunctivitis—Coxsackie A-24 virus (Picornavirus: Enterovirus), Enterovirus 70 (Picornavirus: Enterovirus); Acute hemorrhagic cystitis—Adenovirus 11 and 21 (Adenovirus); AIDS/Acquired Immune Difficiency Syndrome—human immunodeficiency virus (Retrovirus); Anisakidosis—Anisakis simplex (Bacterial) Anthrax—Bacillus anthracis (Bacterial); Aspergilloma/Aspergillosis—Aspergillus (Fungal); Arthritis, Septic—Staphylococcus aureus, or Neisseria gonorrhoeae (Bacterial) Athlete's Foot—Dermatophytes (Fungal); Blastomycosis—Blastomyces dermatitidis (Bacterial) “Black death” (plague)—Yersinia pestis (Bacterial) Bornholm disease (pleurodynia)—Coxsackie B (Picornavirus: Enterovirus) Botulism—Clostridium botulinum (Bacterial) Borna Diease—Borna Diease Virus (Unassigned Virus) Brazilian purpuric fever—Haemophilus aegyptius (Bacterial) Bronchitis—(Bacterial) Bronchiolitis—Respiratory syncytial virus (Paramyxovirus), Parainfluenza virus (Paramyxovirus) Brucellosis—Brucella (Bacterial) Bubonic Plague—Yersinia pestis (Bacterial) California encephalitis—California encephalitis virus (Bunyavirus) Candidiasis—Candida (Yeast) Cat Scratch Fever—Bartonella henselae (Bacterial) Cellulitis—(Bacterial) Cervical cancer—human papilloma virus (Papovavirus) CFS—Chronic Fatigue Syndrome—not an infectious disease Chancroid—Haemophilus ducreyi (Bacterial) Chicken pox—varicella zoster virus (Herpesvirus) Chlamydia—Chlamydiae trachomatis (Bacterial) Cholera—Vibrio cholerae (Bacterial) Chronic Fatigue Syndrome—not an infectious disease Colorado tick fever—Colorado tick fever virus (Reovirus) Conjunctivitis—Haemophilus aegyptius or Chlamydiae trachomatis (Bacterial) or Adenovirus (Adenovirus) or Herpes Simplex Virus (Herpesvirus) Cowpox—vaccinia virus (Poxvirus) Croup, infectious—parainfluenza viruses 1-3 (Paramyxovirus) Cryptosporidiosis—Cryptosporidium parvum or Cryptosporidium coccidi (Protozoan parasite) Darling's Disease—Histoplasma capsulatum (Fungal) Dengue—dengue virus (Flavivirus) Dermatomycoses—Dermatophytes (Fungal) Desert Rheumatism—Coccidioides immites (Bacterial) “Devil's grip”(pleurodynia)—Coxsackie B (Picornavirus: Enterovirus) Diphtheria—Corynebacterium diphtheriae (Bacterial) Dysentery—Shigella (Bacterial) Ear Infection—see Otitis Media Eastern equine encephalitis—EEE virus (Togavirus) Ebola hemorrhagic fever—Ebola virus (Filovirus) Ehrlichiosis—Ehrlichia (Bacterial) Endocarditis—various bacterial pathogens (Bacterial) Epiglottitis—Haemophilus influenzae or Streptococcus pyogenes (Bacterial) Erythema infectiosum—Parvovirus B19 (Parvovirus) “Fifth” disease (erythema infectiosum)—Parvovirus B19 (Parvovirus) “Flesh Eating Bacteria”—Necrotizing fasciitis (NF)—Group A Strep (Bacterial) Food Poisoning—various bacterial pathogens, and some toxins Foot and Mouth Disease (Hand-foot-mouth disease)—Coxsackie A-16 virus (Picornavirus: Enterovirus) Gardener's Disease—Sporothrix schenckii (Fungal) Gas gangrene—Clostridium perfringens (Bacterial) Gastroenteritis—Norwalk virus (Calicivirus), rotavirus (Reovirus), or various bacterial species Genital HSV—Herpes Simples Virus (Herpesvirus) Giardiasis—Giardia lamblia (Protozoan parasite) Gilchrist's Disease—Blastomyces dermatitidis (Fungal) Gingivostomatitis—HSV-1 (Herpesvirus) Gonorrhea—Neisseria gonorrhoeae (Bacterial) Granuloma Inguinale—Calymmatobacterium granulomatis (Bacterial) Hand-foot-mouth disease—Coxsackie A—16 virus (Picornavirus: Enterovirus) Hantavirus hemorrhagic fever/Hantaan-Korean hemorrhagic fever—Hantavirus (Bunyavirus) Hepatitis: Hepatitis A—hepatitis A virus (Picornavirus: Enterovirus) Hepatitis—hepatitis B virus (Hepadnavirus) Hepatitis C—hepatitis C virus (Flavivirus) Hepatitis D—hepatitis D virus (Deltavirus) Hepatitis E—hepatitis E virus (Calicivirus) Herpangina—Coxsackie A (Picornavirus: Enterovirus), Enterovirus 7 (Picornavirus: Enterovirus) Herpes, genital—HSV-2 (Herpesvirus) Herpes labialis—HSV-1 (Herpesvirus) Herpes, neonatal—HSV-2 (Herpesvirus) Histoplasmosis—Histoplasma capsulatum (Fungal) HIV—human immunodeficiency virus (Retrovirus) Impetigo—Streptococcus pyogenes or Staphylococcus aureus (Bacterial) Infectious myocarditis—Coxsackie B1-B5 (Picornavirus: Enterovirus) Infectious pericarditis—Coxsackie B1-B5 (Picornavirus: Enterovirus) Influenza—Influenza viruses A, B, and C (Orthomyxovirus) Japanese encephalitis virus—JEE virus (Flavivirus) Jock Itch—Dermatophytes (Fungal) Junin Argentinian hemorrhagic fever—Juninvirus (Arenavirus) Keratoconjunctivitis—Adenovirus (Adenovirus), HSV-1 (Herpesvirus) Koch-Weeks—see Conjunctivitis LaCrosse encephalitis—LaCross virus (Bunyavirus) Lassa hemorrhagic fever—Lassavirus (Arenavirus) Legionnaire's Disease (Legionnaire's pneumonia)—Legionella pneumophila (Bacterial) Leprosy (Hansen's disease)—Mycobacterium leprae (Bacterial) Leptospirosis—Leptospira interrogans (Spirochetes, Bacterial) Leishmaniasis—Leishmania (Parasitical) Listeriosis—Listeria moncytogenes (Bacterial) Lyme disease—Borrelia burgdoferi (Spirochetes, Bacterial) Machupo Bolivian hemorrhagic fever—Machupovirus (Arenavirus) Malta fever—Brucella sp. (Bacterial) Marburg hemorrhagic fever—Marburg virus (Filovirus) Measles—rubeola virus (Paramyxovirus) Melioidosis—Pseudomonas pseudomallei (Bacterial) Meningitis, aseptic—Coxsackie A and B (Picornavirus: Enterovirus), Echovirus (Picornavirus: Enterovirus), lymphocytic choriomeningitis virus (Arenavirus), HSV-2 (Herpesvirus), Mycobacterium tuberculosis (Bacterial) Meningitis, bacterial—Neisseria meningitidis (Bacterial), Haemophilus influenzae (Bacterial), Listeria monocytogenes (Bacterial), Streptoccoccus pneumoniae, Group B streptococcus (Bacterial) Microsporidiosis—Microsporidia—(single cell Parasites—non-viral) Middle Ear Infection—see Otitis Media Molluscum contagiosum—Molluscum (Poxvirus) Moniliasis—Candida species (Yeast) Mononucleosis—Epstein-Barr virus (Herpesvirus) Mononucleosis-like syndrome—CMV (Herpesvirus) Mumps—mumps virus (Paramyxovirus) Mycotic Vulvovaginitis—Candida species (Yeast—not viral) Necrotizing fasciitis (NF)—Group A Strep (Bacterial) Nocardiosis—Nocardia (Bacterial) Orf—Orfvirus (Poxvirus) Otitis externa—Pseudomonas aeruginosa (Bacterial) Otitis media—Streptococcus pneunomiae, or Haemophilus influenzae, or Moraxella catarrhalis, or Staphylococcus aureus (Bacterial) PCP—Pneumocystis carinii (Bacterial) Pelvic Inflamatory Disease—various Bacterial pathogens (Bacterial) Peritonitis—Escherichia coli, or Bacteriodes (Bacterial) Pertussis—Bordetella pertussis (Bacterial) Phaeohyphomycosis—Dematiaceous Fungi (Fungal) Pharyngoconjunctival fever—Adenovirus 1-3 and 5 (Adenovirus) Pharyngitis: Streptococcus pyogenes (Bacterial) Respiratory Synytial Virus (Paramyxovirus: Pneumovirus) Influenza Virus (Orthomyxovirus) Parainfluenza Virus (Paramyxovirus) Adenovirus (Adenovirus) Epstein-Barr Virus (Herpesvirus) Phycomycosis—Mucor species (Fungal) PID—see Pelvic Inflamatory Disease “Pink eye” conjunctivitis—see Conjunctivitis Plague—Yersinia pestis (Bacterial) Pleurodynia—Coxsackie B (Picornavirus: Enterovirus) Pneumonia, viral—respiratory syncytial virus (Paramyxovirus), CMV (Herpesvirus) Pneumocystis carinii Pneumonia—Pneumocystis carinii (Bacterial) Pneumonic Plague—Yersinia Pestis (Bacterial) Polio, Poliomyelitis—Poliovirus (Picornavirus: Enterovirus) Pontiac fever—Legionella pneumophila (Bacterial) Posadas-Werincke's Disease—Coccidioides immites (Bacterial) Progressive multifocal leukencephalopathy—JC virus (Papovavirus) Pseudomembranous colitis—Clostridium dificile (Bacterial) Psittacosis—Chlamydia psittaci (Bacterial) Q fever—Coxiella burnetti (Rickettsial) Rabies—rabies virus (Rhabdovirus) Red Eye—see Conjunctivitis Reticuloendotheliosis—Histoplasma capsulatum (Bacterial) Rheumatic Fever—Streptococcus pyogenes (Bacterial) Ring Worm—Dermatophytes (Fungal) Rocky Mountain Spotted Fever (RMSF)—Rickettsia rickettsii (Rickettsial) Roseola—HHV-6 (Herpesvirus) Rubella—rubivirus (Togavirus) Rubeola—see Measles Salmonellosis—Salmonella species (Bacterial) San Joaquin Fever—Coccidioides immitis (Bacterial) Scabies—Sarcoptes scabiei (Mites) Scarlet fever—Streptococcus pyogenes (Bacterial) Schistosomiasis—Schistosomiasis mansoni (Bacterial) Sepsis—various Bacterial Pathogens (Bacterial) Septic Arthritis—Staphylococcus aureus, or Neisseria gonorrhoeae (Bacterial) Septic Thrombophlebitis—see Thrombophlebitis Shigellosis—Shigella species (Bacterial) Shingles (zoster)—varicella zoster virus (Herpesvirus) Shipping fever—Pasteurella multocida (Bacterial) Sinusitis—various Bacterial Pathogens (Bacterial) Smallpox—variola virus (Poxvirus) “Slapped cheek” disease (erythema infectiosum)—Parvovirus B19 (Parvovirus) Sporotrichosis—Sporothrix schenckii (Fungal) St. Louis encephalitis—SLE virus (Flavivirus) Strep Throat—see Pharyngitis Strongyloidiasis—Strongyloides stercoralis (Bacterial) Swimmer's Ear—See Otitis Extema Syphilis—Treponema pallidum (Spirochete bacteria) Temporal lobe encephalitis—HSV-1 (Herpesvirus) Tetanus—Clostridium tetani (Bacterial) Thrombophlebitis—Staphylococcus species (Bacterial) Thrush—Candida species (Yeast) Tinea—Dermatophytes (Fungal) Toxic Shock Syndrome—Staphylococcus aureus or Streptococcus pyogenes (Bacterial) Toxoplasmosis—Toxoplasma gondii (Sporozoan) Trachoma—Chlamydia trachomatis (Bacterial) Trichinosis—Trichinella spiralis (Nematode) Trichomoniasis—Trichomonas vaginalis (Protozoan) Tuberculosis—Mycobacterium tuberculosis (Bacterial) Tularemia—Francisella tularensis (Bacterial) Typhoid fever—Salmonella typhi (Bacterial) Undulating fever—Brucella species (Bacterial) Urinary Tract Infection (UTI)—various Bacterial Pathogens (Bacterial) Urethritis—Chlamydia trachomatis (Bacterial), or Trichomonas vaginalis (Protozoan), or Herpes Simples Virus (Herpesvirus), Ureaplasma urealyticum (Mycoplasma) Vaginosis—Gardnerella vaginalis (Bacterial), or Bacteroides species (Bacterial), or Streptococcus species (Bacterial) Valley Fever—Coccidioides immitis (Bacterial) Varicella—varicella zoster virus (Herpesvirus) Vulvovaginitis, Mycotic—Candida species (Yeast—not viral) Western equine encephalitis—WEE virus (Togavirus) Whooping Cough—Bordetella pertussis (Bacterial) Wool sorters' disease—Bacillus anthracis (Bacterial) Yellow fever—Yellow fever virus (Flavivirus) Zoster—varicella zoster virus (Herpesvirus) Zygomycosis—Mucor species (Fungal)

The following table (Table 1) also shows particular virus families that may detected by the methods of the present invention.

TABLE 1

| Genome & Physical Characteristics | | | Virus Family |
|---|---|---|---|
| Type | Nucleic Acid Description | Envelope | Name |
| DNA | ds | enveloped | Baculoviridae |
| | | | Herpesviridae |
| | | | Iridoviridae |
| | | | Poxviridae |
| | | | “African Swine Fever |
| | | | Viruses” |
| | | | (unnamed family) |
| | | nonenveloped | Adenoviridae |
| | | | Caulimoviridae |
| | | | Myrovidae |
| | | | Phycodnaviridae |
| | | | Tectiviridae |
| | | | Papovaviridae |
| | ss | nonenveloped | Circoviridae |
| | | | Parvoviridae |
| | ds/ss | enveloped | Hepadnaviridae |
| RNA | positive | | |
| | ds | | |
| | nonsegmented | enveloped | Cystoviridae |
| | segmented | nonenveloped | Birnaviridae |
| | | | Reoviridae |
| | ss | | |
| | nonsegmented | enveloped | Coronaviridae |
| | | | Flaviviridae |
| | | | Togaviridae |
| | | | “Arterivirus” |
| | | | (a floating genus) |
| | | nonenveloped | Astroviridae |
| | | | Caliciviridae |
| | | | Picornaviridae |
| | | | Potyviridae |
| | DNA step in replication | enveloped | Retroviridae |
| | negative | | |
| | segmented | enveloped | Orthomyxoviridae |
| | nonsegmented | enveloped | Filoviridae |
| | | | Paramyxoviridae |
| | | | Rhabdoviridae |
| | negative & | | |
| | ambisense | | |
| | segmented | enveloped | Arenaviridae |
| | | | Bunyaviridae |

| Partially Assigned Viruses: | |
|---|---|
| Genome & Physical Characteristics | Virus Genus |
| ssRNA, nonenveloped | Tobamovirus |
| | Carlavirus |

The present invention also relates to detecting target nucleic acid sequences (and mutations therein) in pooled nucleic acid samples (e.g. in a pooled biological sample from a plurality of donors). In particular, the present invention relates to compositions and methods for detecting target nucleic acid sequences, mutations, or measuring allele frequencies in pooled nucleic acid samples employing the detection assay (e.g. INVADER assay). In some embodiments, the present invention provides methods for detecting an allele frequency of a polymorphism, comprising: a) providing; i) a pooled sample, wherein the pooled sample comprises target nucleic acid sequences from at least 10 individuals (or at least 50, or at least 100, or at least 250, or at least 500, or at least 1000 individuals, etc.); and ii) INVADER assay reagents (e.g. primary probes, INVADER oligonucleotides, FRET cassettes, a structure specific enzyme, etc.) configured to detect the presence or absence of a polymorphism; and b) contacting the pooled sample with the INVADER assay (detection) reagents to generate a detectable signal; and c) measuring the detectable signal, thereby determining a number of the target nucleic acid sequences that contain the polymorphism (e.g. a quantitative number of molecules, or the allele frequency for the polymorphism in a population, is determined). In some embodiments, signals from two or more alleles for a particular target nucleic acid locus are measured and the numbers are compared. In preferred embodiments, the measurements for two or more different alleles of a particular target nucleic acid locus are measured in a single reaction. In other embodiments, measurements from one or more alleles of a particular target nucleic acid locus are compared to measurements from one or more reference target nucleic acid loci. In preferred embodiments, measurements from one or more alleles of a particular target nucleic acid locus are compared to measurements from one or more reference target nucleic acid loci in the same reaction mixture. Further methods allow a single individual's particular allele frequency (i.e., frequency of the mutation among multiple copies of the sequence within an individual) or quantitative number of molecules found to possess the polymorphism (e.g. determined by an INVADER assay) to be compared to the population allele frequency (or expected number), such that it is determined if the single individual is susceptible to a disease, how far a disease has progressed (e.g. diseases such as cancer that may be diagnosed by identifying loss of heterozygosity), etc. In some embodiments, the individuals are from the same racial or ethnic class (e.g. European, African, Asian, Mexican, etc).

In particular embodiments, the present invention provides methods for detecting a rare mutation comprising; a) providing; i) a sample from a single subject, wherein the sample comprises at least 10,000 target nucleic acid sequences (e.g. from 10,000 cells, or at least 20,000 target nucleic acid sequences, or at least 100,000 target nucleic acid sequences), ii) a detection assay (e.g. the INVADER assay) capable of detecting a mutation in a population of target nucleic acid sequence that is present at an allele frequency of 1:1000 or less compared to wild type alleles; and b) assaying the sample with the detection assay under conditions such that the presence or absence of a rare mutation (e.g. one present at an allele frequency of 1:100, or 1:500, or 1:1000 or less compared to the wild type) is detected. In some embodiments, the target nucleic acid sequences are genomic (e.g. not polymerase chain reaction, or PCR, amplified, but directly from a cell). In other embodiments, the target nucleic acid sequences are amplified (e.g., by PCR).

In some embodiments, the present invention provides methods for detecting a rare mutation comprising; a) providing; i) a sample from a single subject, wherein the sample comprises at least 10,000 target nucleic acid sequences, ii) a detection assay capable of detecting a mutation in a population of target nucleic acid sequence that is present at an allele frequency of 1:1000 or less compared to wild type alleles; and b) assaying the sample with the detection assay under conditions such that an allele frequency in the sample of a rare mutation is determined. In some embodiments, the subject's allele frequency is compared statistically to a known reference allele frequency (e.g. determined by the methods of the present invention or other methods), such that a diagnosis may be made (e.g. extent of disease, likelihood of having the disease, or passing it on to offspring, etc).

The present invention also provides methods for determining the number of molecules of one or more polymorphisms present in a sample by employing, for example, the INVADER assay (e.g. polymorphisms such as SNPs that are associated with disease). This assay may be used to determine the number of a particular polymorphism in a first sample, and then determining if there is a statistically-significant difference between that number and the number of the same polymorphism in a second sample. Preferably, one sample represents the number of the polymorphism expected to occur in a sample obtained from a healthy individual, or from a healthy population if pooled samples are used. A statistically significant difference between the number of a polymorphism expected to be at a single-base locus in a healthy individual and the number determined to be in a sample obtained from a patient is clinically indicative.

### DESCRIPTION OF THE FIGURES

FIG. 1 shows a schematic of the INVADER assay. While this figure shows the wild type probe causing a detectable signal (while the mutant probe does not cause a signal), it is understood that the INVADER assay may also be configured such that it is the mutant probe that causes a signal to be generated (while the wild type probe does not cause a signal).

FIG. 2 shows a graph demonstrating the ability of the INVADER assay to detect mutations in the APOC4 gene in pooled samples.

FIG. 3 shows a graph demonstrating the ability of the INVADER assay to detect mutations in the CFTR gene in pooled samples.

FIGS. 4a-c show graphs of the results of experiments described in Example 3.

FIG. 5A shows data measuring allele signals in INVADER assays for detection of alleles comprising the indicated percentages of the number of copies of each locus.

FIG. 5B shows an Excel graph comparing theoretical allele frequencies to allele frequencies calculated from the INVADER assay data shown in FIG. 5A.

FIG. 6 shows an Excel graph and data comparing actual and calculated allele frequencies for each of 8 SNP loci detected in pooled genomic DNA from 8 different individuals.

FIG. 7A shows an Excel graph and data showing calculated allele frequencies compared to fold-over-zero minus 1 (FOZ-1) measurements for SNP locus 132505 in genomic DNAs having different mixtures of these alleles.

FIG. 7B shows an Excel graph and data showing calculated allele frequencies compared to fold-over-zero minus 1 (FOZ-1) measurements for SNP locus 131534 in genomic DNAs having different mixtures of these alleles.

FIGS. 8A-8C show the sequences of the probes configured for use in the assays described in Example 4 and synthetic targets for each allele. “Y” indicates an amine blocking group. The polymorphism and the dye that will be detected for each probe, when used in the exemplary assay configurations described in Example 4, are indicated.

FIG. 9 shows a schematic diagram of four sets of INVADER assay oligonucleotides aligned on a portion of HIV transcript 3. Each set comprises a probe, a stacker and an INVADER oligonucleotide. The probe, stacker and INVADER oligonucleotides of Set 1 are SEQ ID NOS: 113, 120, and 117, respectively; for Sets 2 and 4, the stacker and INVADER oligonucleotides are SEQ ID NOS: 121 and 118, respectively, with Set 2 using probe oligonucleotide SEQ ID NO: 114 and set 4 using probe oligonucleotide SEQ ID NO: 116; The probe, stacker and INVADER oligonucleotides of Set 3 are SEQ ID NOS: 115, 118 and 119, respectively.

FIG. 10 shows probe turnover rates (min˜′) as determined in the INVADER assay for each of the probe sets shown in FIG. 9, and the effects of using the sets without or with the corresponding stacker oligonucleotide.

FIG. 11 shows a schematic diagram of an INVADER oligonucleotide (SEQ ID NO:117), primary probe oligonucleotide (SEQ ID NO:122), a stacker oligonucleotide (SEQ ID NO:120), an ARRESTOR oligonucleotide (SEQ ID NO:123), a secondary target oligonucleotide (SEQ ID NO:125) and FRET probe (SEQ ID NO:126) for the detection of HIV RNA. The primary probe and INVADER oligonucleotides are shown aligned with a portion of HIV transcript 3. Cleavage of the primary probe oligonucleotide produces the arm oligonucleotide having SEQ ID NO:124.

FIG. 12 shows the accumulated fluorescence signal from INVADER assay reactions using the oligonucleotides diagrammed in FIG. 11, over a range of concentrations of HIV viral RNA. Target copy number is indicated in copies per reaction.

FIG. 13 shows a schematic diagram of an INVADER oligonucleotide (SEQ ID NO:128), primary probe oligonucleotide (SEQ ID NO:129), a stacker oligonucleotide (SEQ ID NO:127), an ARRESTOR oligonucleotide (SEQ ID NO:130), a secondary target oligonucleotide (SEQ ID NO:125) and FRET probe (SEQ ID NO:126) for the detection of HIV RNA. The primary probe and INVADER oligonucleotides are shown aligned with a portion of HIV transcript 3. Cleavage of the primary probe oligonucleotide produces the arm oligonucleotide having SEQ ID NO:124.

FIG. 14 shows the accumulated fluorescence signal from INVADER assay reactions using the oligonucleotides diagrammed in FIG. 13, over a range of concentrations of HIV viral RNA. Target copy number is indicated in copies per reaction.

FIG. 15 shows the results of Example 6.

### DEFINITIONS

To facilitate an understanding of the invention, a number of terms are defined below.

As used herein, the terms “subject” and “patient” refer to any organisms including plants, microorganisms and animals (e.g., mammals such as dogs, cats, livestock, and humans).

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “variant” or “mutant” or “mutation” refer to a gene or gene product that displays modifications in sequence and/or functional properties (i.e., altered characteristics) when compared to the wild-type gene or gene product.

As used herein, the term “amplifiable nucleic acid” is used in reference to nucleic acids that may be amplified by any amplification method. It is contemplated that “amplifiable nucleic acid” will usually comprise a nucleic acid “target region” or “sample template.”

As used herein, the term “sample template” refers to nucleic acid originating from a sample that is analyzed for the presence of “target” or “target nucleic acid sequence” (defined below). In contrast, “background template” is used in reference to nucleic acid other than sample template that may or may not be present in a sample. Background template may be inadvertent. It may be the result of carryover, or it may be due to the presence of nucleic acid contaminants sought to be purified away from the sample. For example, nucleic acids from organisms other than those to be detected may be present as background in a test sample. Background template may also be the region of a nucleic acid containing the wild type allele.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer should be sufficiently long to prime the synthesis of extension products in the presence of the inducing agent. The exact lengths of the primers will depend on many factors, including temperature, source of primer and the use of the method.

As used herein, the term “target,” or “target nucleic acid sequence” refers to a nucleic acid sequence or structure to be detected or characterized. Thus, the “target” or “target nucleic acid sequence” is sought to be sorted out or identified from other nucleic acid sequences. Examples of “targets” or “target nucleic acid sequences” include, but are not limited to, viral RNA sequences (e.g. from HCV or HIV).

As used herein, the term “polymerase chain reaction” (“PCR”) refers to the method described in U.S. Pat. Nos. 4,683,195, 4,683,202, and 4,965,188, hereby incorporated by reference, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein the terms “portion” or “region” when in reference to a nucleotide sequence (as in “a portion of a given nucleotide sequence”) refer to fragments of that sequence. The fragments may range in size from four nucleotides to the entire nucleotide sequence minus one nucleotide (10 nucleotides, 20, 30, 40, 50, 100, 200, etc.).

As used herein, the term “sample” is used in its broadest sense to include any material that may be tested.

As used herein, the term “rare mutation” refers to a mutation that is present in 20% or less (preferably 10% or less, more preferably 5% or less, and more preferably 1% or less) of a population of nucleic acid molecules in a sample (i.e., wherein the remaining 80% or more of the nucleic acid molecules have a wild type sequence or a different mutation in the corresponding region of the nucleic acid molecules).

As used herein, the term “distinct” in reference to signals refers to signals that can be differentiated one from another, e.g., by spectral properties such as fluorescence emission wavelength, color, absorbance, mass, size, fluorescence polarization properties, charge, etc., or by capability of interaction with another moiety, such as with a chemical reagent, an enzyme, an antibody, etc.

As used herein, the term “INVADER assay reagents” refers to one or more reagents for detecting target sequences, said reagents comprising oligonucleotides capable of forming an invasive cleavage structure in the presence of the target sequence. In some embodiments, the INVADER assay reagents further comprise an agent for detecting the presence of an invasive cleavage structure (e.g., a cleavage agent). In some embodiments, the oligonucleotides comprise first and second oligonucleotides, said first oligonucleotide comprising a 5′ portion complementary to a first region of the target nucleic acid and said second oligonucleotide comprising a 3′ portion and a 5′ portion, said 5′ portion complementary to a second region of the target nucleic acid downstream of and contiguous to the first portion. In some embodiments, the 3′ portion of the second oligonucleotide comprises a 3′ terminal nucleotide not complementary to the target nucleic acid. In preferred embodiments, the 3′ portion of the second oligonucleotide consists of a single nucleotide not complementary to the target nucleic acid.

In some embodiments, INVADER assay reagents are configured to detect a target nucleic acid sequence comprising first and second non-contiguous single-stranded regions separated by an intervening region comprising a double-stranded region. In preferred embodiments, the INVADER assay reagents comprise a bridging oligonucleotide capable of binding to said first and second non-contiguous single-stranded regions of a target nucleic acid sequence. In particularly preferred embodiments, either or both of said first or said second oligonucleotides of said INVADER assay reagents are bridging oligonucleotides.

In some embodiments, the INVADER assay reagents further comprise a solid support. For example, in some embodiments, the one or more oligonucleotides of the assay reagents (e.g., first and/or second oligonucleotide, whether bridging or non-bridging) is attached to said solid support. In some embodiments, the INVADER assay reagents further comprise a buffer solution. In some preferred embodiments, the buffer solution comprises a source of divalent cations (e.g., Mn²⁺ and/or Mg²⁺ ions). Individual ingredients (e.g., oligonucleotides, enzymes, buffers, target nucleic acids) that collectively make up INVADER assay reagents are termed “INVADER assay reagent components”.

In some embodiments, the INVADER assay reagents further comprise a third oligonucleotide complementary to a third portion of the target nucleic acid upstream of the first portion of the first target nucleic acid. In yet other embodiments, the INVADER assay reagents further comprise a target nucleic acid. In some embodiments, the INVADER assay reagents further comprise a second target nucleic acid. In yet other embodiments, the INVADER assay reagents further comprise a third oligonucleotide comprising a 5′ portion complementary to a first region of the second target nucleic acid. In some specific embodiments, the 3′ portion of the third oligonucleotide is covalently linked to the second target nucleic acid. In other specific embodiments, the second target nucleic acid further comprises a 5′ portion, wherein the 5′ portion of the second target nucleic acid is the third oligonucleotide. In still other embodiments, the INVADER assay reagents further comprise an ARRESTOR molecule (e.g., ARRESTOR oligonucleotide).

In some preferred embodiments, the INVADER assay reagents further comprise reagents for detecting a nucleic acid cleavage product. In some embodiments, one or more oligonucleotides in the INVADER assay reagents comprise a label. In some preferred embodiments, said first oligonucleotide comprises a label. In other preferred embodiments, said third oligonucleotide comprises a label. In particularly preferred embodiments, the reagents comprise a first and/or a third oligonucleotide labeled with moieties that produce a fluorescence resonance energy transfer (FRET) effect.

In some embodiments one or more the INVADER assay reagents may be provided in a predispensed format (i.e., premeasured for use in a step of the procedure without re-measurement or re-dispensing). In some embodiments, selected INVADER assay reagent components are mixed and predispensed together. In other embodiments, In preferred embodiments, predispensed assay reagent components are predispensed and are provided in a reaction vessel (including but not limited to a reaction tube or a well, as in, e.g., a microtiter plate). In particularly preferred embodiments, predispensed INVADER assay reagent components are dried down (e.g., desiccated or lyophilized) in a reaction vessel.

In some embodiments, the INVADER assay reagents are provided as a kit. As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

In some embodiments, the present invention provides INVADER assay reagent kits comprising one or more of the components necessary for practicing the present invention. For example, the present invention provides kits for storing or delivering the enzymes and/or the reaction components necessary to practice an INVADER assay. The kit may include any and all components necessary or desired for assays including, but not limited to, the reagents themselves, buffers, control reagents (e.g., tissue samples, positive and negative control target oligonucleotides, etc.), solid supports, labels, written and/or pictorial instructions and product information, inhibitors, labeling and/or detection reagents, package environmental controls (e.g., ice, desiccants, etc.), and the like. In some embodiments, the kits provide a sub-set of the required components, wherein it is expected that the user will supply the remaining components. In some embodiments, the kits comprise two or more separate containers wherein each container houses a subset of the components to be delivered. For example, a first container (e.g., box) may contain an enzyme (e.g., structure specific cleavage enzyme in a suitable storage buffer and container), while a second box may contain oligonucleotides (e.g., INVADER oligonucleotides, probe oligonucleotides, control target oligonucleotides, etc.).

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxgenin; luminogenic, phosphorescent or fluorogenic moieties; mass tags; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, characteristics of mass or behavior affected by mass (e.g., MALDI time-of-flight mass spectrometry), and the like. A label may be a charged moiety (positive or negative charge) or alternatively, may be charge neutral. Labels can include or consist of nucleic acid or protein sequence, so long as the sequence comprising the label is detectable.

### DESCRIPTION OF THE INVENTION

The present invention relates to detecting target nucleic acid sequences in pooled samples. In particular, the present invention relates to compositions and methods for detecting the presence or absence of target nucleic acid sequences (e.g. RNA virus sequences) in a pooled sample employing an INVADER detection assay.

I. INVADER Assays and Pooled Samples

The INVADER assay detects hybridization of probes to a target by enzymatic cleavage of specific structures by structure specific enzymes (See, INVADER assays, Third Wave Technologies; See e.g., U.S. Pat. Nos. 5,846,717; 6,090,543; 6,001,567; 5,985,557; 6,090,543; 5,994,069; Lyamichev et al., Nat. Biotech., 17:292 (1999), Hall et al., PNAS, USA, 97:8272 (2000), WO97/27214 and WO98/42873, each of which is herein incorporated by reference in their entirety for all purposes).

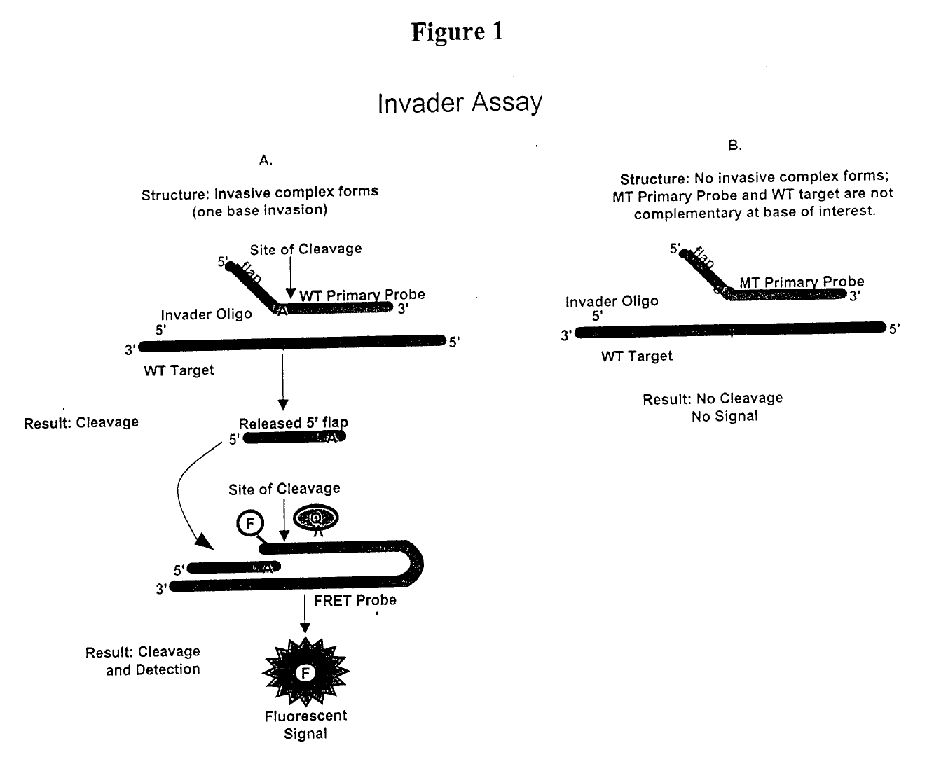

The INVADER assay detects specific DNA and RNA sequences by using structure-specific enzymes (e.g. FEN endonucleases) to cleave a complex formed by the hybridization of overlapping oligonucleotide probes (See, e.g. FIG. 1). Elevated temperature and an excess of one of the probes enable multiple probes to be cleaved for each target sequence present without temperature cycling. In some embodiments, these cleaved probes then direct cleavage of a second labeled probe. The secondary probe oligonucleotide can be 5′-end labeled with fluorescein that is quenched by an internal dye. Upon cleavage, the de-quenched fluorescein labeled product may be detected using a standard fluorescence plate reader.

The INVADER assay detects specific mutations and SNPs in unamplified, as well as amplified, RNA and DNA including genomic DNA. In the embodiments shown schematically in FIG. 1, the INVADER assay uses two cascading steps (a primary and a secondary reaction) both to generate and then to amplify the target-specific signal. For convenience, the alleles in the following discussion are described as wild-type (WT) and mutant (MT), even though this terminology does not apply to all genetic variations. In the primary reaction (FIG. 1, panel A), the WT primary probe and the INVADER oligonucleotide hybridize in tandem to the target nucleic acid to form an overlapping structure. An unpaired “flap” is included on the 5′ end of the WT primary probe. A structure-specific enzyme (e.g. the CLEAVASE enzyme, Third Wave Technologies) recognizes the overlap and cleaves off the unpaired flap, releasing it as a target-specific product. In the secondary reaction, this cleaved product serves as an INVADER oligonucleotide on the WT fluorescence resonance energy transfer (WT-FRET) probe to again create the structure recognized by the structure specific enzyme (panel A). When the two dyes on a single FRET probe are separated by cleavage (indicated by the arrow in FIG. 1), a detectable fluorescent signal above background fluorescence is produced. Consequently, cleavage of this second structure results in an increase in fluorescence, indicating the presence of the WT allele (or mutant allele if the assay is configured for the mutant allele to generate the detectable signal). In some embodiments, FRET probes having different labels (e.g. resolvable by difference in emission or excitation wavelengths, or resolvable by time-resolved fluorescence detection) are provided for each allele or locus to be detected, such that the different alleles or loci can be detected in a single reaction. In such embodiments, the primary probe sets and the different FRET probes may be combined in a single assay, allowing comparison of the signals from each allele or locus in the same sample.

If the primary probe oligonucleotide and the target nucleotide sequence do not match perfectly at the cleavage site (e.g., as with the MT primary probe and the WT target, FIG. 1, panel B), the overlapped structure does not form and cleavage is suppressed. The structure specific enzyme (e.g. CLEAVASE VIII enzyme, Third Wave Technologies) used cleaves the overlapped structure more efficiently (e.g. at least 340-fold) than the non-overlapping structure, allowing excellent discrimination of the alleles.

The probes turn over without temperature cycling to produce many signals per target (i.e., linear signal amplification). Similarly, each target-specific product can enable the cleavage of many FRET probes.

The primary INVADER assay reaction is directed against the target DNA (or RNA) being detected. The target DNA is the limiting component in the first invasive cleavage, since the INVADER and primary probe are supplied in molar excess. In the second invasive cleavage, it is the released flap that is limiting. When these two cleavage reactions are performed sequentially, the fluorescence signal from the composite reaction accumulates linearly with respect to the target DNA amount.

In certain embodiments, the INVADER assay, or other nucleotide detection assays, are performed with accessible site designed oligonucleotides and/or bridging oligonucleotides. Such methods, procedures and compositions are described in U.S. Pat. No. 6,194,149, WO9850403, and WO0198537, all of which are specifically incorporated by reference in their entireties.

In certain embodiments, the target nucleic acid sequence is amplified prior to detection (e.g. such that synthetic nucleic acid is generated). In some embodiments, the target nucleic acid comprises genomic DNA. In other embodiments, the target nucleic acid comprises synthetic DNA or RNA. In some preferred embodiments, synthetic DNA within a sample is created using a purified polymerase. In some preferred embodiments, creation of synthetic DNA using a purified polymerase comprises the use of PCR. In other preferred embodiments, creation of synthetic DNA using a purified DNA polymerase, suitable for use with the methods of the present invention, comprises use of rolling circle amplification, (e.g., as in U.S. Pat. Nos. 6,210,884, 6,183,960 and 6,235,502, herein incorporated by reference in their entireties). In other preferred embodiments, creation of synthetic DNA comprises copying genomic DNA by priming from a plurality of sites on a genomic DNA sample. In some embodiments, priming from a plurality of sites on a genomic DNA sample comprises using short (e.g., fewer than about 8 nucleotides) oligonucleotide primers. In other embodiments, priming from a plurality of sites on a genomic DNA comprises extension of 3′ ends in nicked, double-stranded genomic DNA (i.e., where a 3′ hydroxyl group has been made available for extension by breakage or cleavage of one strand of a double stranded region of DNA). Some examples of making synthetic DNA using a purified polymerase on nicked genomic DNAs, suitable for use with the methods and compositions of the present invention, are provided in U.S. Pat. Nos. 6,117,634, issued Sep. 12, 2000, and 6,197,557, issued Mar. 6, 2001, and in PCT application WO 98/39485, each incorporated by reference herein in their entireties for all purposes.

In some embodiments, the present invention provides methods for detecting a target sequence, comprising: providing a) a sample containing DNA amplified by extension of 3′ ends in nicked double-stranded genomic DNA, said genomic DNA suspected of containing said target sequence; b) oligonucleotides capable of forming an invasive cleavage structure in the presence of said target sequence; and c) exposing the sample to the oligonucleotides and the agent. In some embodiments, the agent comprises a cleavage agent. In some particularly preferred embodiments, the method of the invention further comprises the step of detecting said cleavage product.

In some preferred embodiments, the exposing of the sample to the oligonucleotides and the agent comprises exposing the sample to the oligonucleotides and the agent under conditions wherein an invasive cleavage structure is formed between said target sequence and said oligonucleotides if said target sequence is present in said sample, wherein said invasive cleavage structure is cleaved by said cleavage agent to form a cleavage product.

In some particularly preferred embodiments, the target sequence comprises a first region and a second region, said second region downstream of and contiguous to said first region, and said oligonucleotides comprise first and second oligonucleotides, said wherein at least a portion of said first oligonucleotide is completely complementary to said first portion of said target sequence and wherein said second oligonucleotide comprises a 3′ portion and a 5′ portion, wherein said 5′ portion is completely complementary to said second portion of said target nucleic acid.

In some embodiments, the target sequence includes, but is not limited to, human cytomegalovirus viral DNA; polymorphisms in human apolipoprotein E gene; mutations in human hemochromatosis gene; mutations in human MTHFR; prothrombin 20210GA polymorphism; HR-2 mutation in human factor V gene; single nucleotide polymorphisms in human TNF-α gene, and Leiden mutation in human factor V gene.

In other embodiments, synthetic DNA suitable for use with the methods and compositions of the present invention is made using a purified polymerase on multiply-primed genomic DNA, as provided, e.g., in U.S. Pat. Nos. 6,291,187, and 6,323,009, and in PCT applications WO 01/88190 and WO 02/00934, each herein incorporated by reference in their entireties for all purposes. In these embodiments, amplification of DNA such as genomic DNA is accomplished using a DNA polymerase, such as the highly processive Φ29 polymerase (as described, e.g., in U.S. Pat. Nos. 5,198,543 and 5,001,050, each herein incorporated by reference in their entireties for all purposes) in combination with exonuclease-resistant random primers, such as hexamers.

In some embodiments, the present invention provides methods for detecting a target sequence, comprising: providing a) a sample containing DNA amplified by extension of multiple primers on genomic DNA, said genomic DNA suspected of containing said target sequence; b) oligonucleotides capable of forming an invasive cleavage structure in the presence of said target sequence; and c) exposing the sample to the oligonucleotides and the agent. In some embodiments, the agent comprises a cleavage agent. In some preferred embodiments, said primers are random primers. In particularly preferred embodiments, said primers are exonuclease resistant. In some particularly preferred embodiments, the method of the invention further comprises the step of detecting said cleavage product.

In some preferred embodiments, the exposing of the sample to the oligonucleotides and the agent comprises exposing the sample to the oligonucleotides and the agent under conditions wherein an invasive cleavage structure is formed between said target sequence and said oligonucleotides if said target sequence is present in said sample, wherein said invasive cleavage structure is cleaved by said cleavage agent to form a cleavage product.

In some preferred embodiments, the exposing of the sample to the oligonucleotides and the agent comprises exposing the sample to the oligonucleotides and the agent under conditions wherein an invasive cleavage structure is formed between said target sequence and said oligonucleotides if said target sequence is present in said sample, wherein said invasive cleavage structure is cleaved by said cleavage agent to form a cleavage product.

In some particularly preferred embodiments, the target sequence comprises a first region and a second region, said second region downstream of and contiguous to said first region, and said oligonucleotides comprise first and second oligonucleotides, said wherein at least a portion of said first oligonucleotide is completely complementary to said first portion of said target sequence and wherein said second oligonucleotide comprises a 3′ portion and a 5′ portion, wherein said 5′ portion is completely complementary to said second portion of said target nucleic acid.

In some embodiments, the target sequence includes, but is not limited to, human cytomegalovirus viral DNA; polymorphisms in human apolipoprotein E gene; mutations in human hemochromatosis gene; mutations in human MTHFR; prothrombin 20210GA polymorphism; HR-2 mutation in human factor V gene; single nucleotide polymorphisms in human TNF-α gene, and Leiden mutation in human factor V gene.

In certain embodiments, the present invention provides kits for assaying a pooled sample (e.g. pooled blood sample) using INVADER detection reagents (e.g. primary probe, INVADER probe, and FRET cassette). In preferred embodiments, the kit further comprises instructions on how to perform the INVADER assay and specifically how to apply the INVADER detection assay to pooled samples from many individuals, or to “pooled” samples from many cells (e.g. from a biopsy sample) from a single subject.

In particular embodiments, the present invention allows detection of target nucleic acid sequences and polymorphims in pooled samples combined from many individuals in a population (e.g. 10, 50, 100, or 500 individuals), or from a single subject where the nucleic acid sequences are from a large number of cells that are assayed at once. In this regard, the present invention allows the frequency of rare mutations in pooled samples to be detected and an allele frequency for the population established. In some embodiments, this allele frequency may then be used to statistically analyze the results of applying the INVADER detection assay to an individual's frequency for the polymorphism (e.g. determined using the INVADER assay). In this regard, mutations that rely on a percent of mutants found (e.g. loss of heterozygozity mutations) may be analyzed, and the severity of disease or progression of a disease determined (See, e.g. U.S. Pat. Nos. 6,146,828 and 6,203,993 to Lapidus, hereby incorporated by reference for all purposes, where genetic testing and statistical analysis are employed to find disease causing mutations or identify a patient sample as containing a disease causing mutations).

In some embodiments of the present invention, broad population screens are performed. In some preferred embodiments, pooling DNA from several hundred or a thousand individuals is optimal. In such a pool, for example, DNA from any one individual would not be detectable, and any detectable signal would provide a measure of frequency of the detected allele in a broader population. The amount of DNA to be used, for example, would be set not by the number of individuals in a pool, as was done in the 15-person pool described in Example 3, but rather by the allele frequency to be detected. For example, the assay in the 96-well format would give ample signal from 20 to 40 ng of DNA in a 90 minute reaction. At this level of sensitivity, analysis of 1 μg of DNA from a high-complexity pool would produce comparable signal from alleles present in only about 3-5% of the population. In some embodiments, reactions are configured to run in smaller volumes, such that less DNA is required for each analysis. In some preferred embodiments, reactions are performed in microwell plates (e.g., 384-well assay plates), and at least two alleles or loci are detected in each reaction well. In particularly preferred embodiments, the signals measured from each of said two or more alleles or loci in each well are compared.

II. Nucleic Acid Detection in Pooled Biological Samples

In preferred embodiments, the present invention provides methods for detecting target nucleic acid sequences (and polymorphisms therein) in pooled biological samples. Examples of pooled biological samples include pooled urine, semen, blood, and blood plasma samples. In certain embodiments, the pooled biological samples are generated by taking a portion of plurality of individual biological samples (e.g. from multiple individuals) and combining these sample into a pool. This pool can then be tested (e.g. for the presence of viral RNA/DNA) more economically as compared to testing each individual biological sample separately.

Pooled blood plasma sample testing has been used for many years. Blood plasma is the yellowish, protein-rich fluid that suspends the cellular components of whole blood. Plasma is a very complex and not fully understood mixture of proteins that performs and enables many housekeeping and other specialized bodily functions. In blood plasma, the most abundant protein is albumin, which makes up approximately 32 to 35 grams per liter. Blood plasma is collected by blood banks and blood collection facilities in two primary ways. One way is plasma is separated from donor collected whole blood. A second way is from donated plasma, in a process where whole blood is drawn from a donor, the plasma is separated, and the remainder is returned to the donor.

Plasma-based products are manufactured from batches of blood plasma collected from many thousands of blood donors. The processing of one pooled lot of plasma can take up to six months, and because of the concerns of infections agents, by rule, the process begins with a 90-day quarantine period. Unlike cellular blood components, products derived from plasma can be treated with chemicals, heat, UV light or filtration to decrease cost and increase ease of handling and distribution, and to increase the safety of the material. Each of these methods have certain drawback that may leave unsafe levels of certain agents (such as viruses), are costly, and/or damage the blood plasma.

One method currently employed intended to economically treat blood plasma and ensure its safety is based on plasma pooling (e.g. pooling 2,000 to tens of thousands of plasma samples), and treating with a solvent detergent. One FDA approved process involves treating the blood plasma pool with tri-N-butyl phosphate (TNBP) and detergent Triton X-100 in an effort to destroy lipid bound viruses such as HIV1 and 2, HCV, HBV, and HTLV I and II. This process does not destroy non-envoloped viruses such as parvovirus, hepatitis A virus, or any prior particles. This detergent process may include the pooling of up to 500,000 individual units of thawed fresh froze blood plasma. After treatment, the plasma is generally sterile filtered and repackaged into approximately 200 mL aliquots or bags and re-frozen.

Disadvantages of the large scale pooling (2,000 to tens of thousands) is the fact that the washing technique does not destroy certain viruses, and the fact that certain batches may not be thoroughly disinfected, thereby contaminating a large pool of plasma. The methods of the present invention identify and prevent such contamination. For example, the INVADER assay may be used to detect all types of viruses in pools or sub-pools prior to being added to giant (2,000 to tens of thousands) pool (e.g. before or after detergent cleaning). In this regard, the present invention provides improved methods for testing and improving the safety of blood plasma.

The American red cross in March 1999, began nucleic acid testing (NAT) in nine of its Blood Services regions. The Red Cross initially used NAT to screen donors for HCV and HIV-1. While the risk of those viruses in volunteer donors is very low, the Red Cross conducted studies to see if NAT could further reduce the “window period” for donor blood. The window period is the length of time after infection that it takes to detect antigens to the virus or for a person to develop enough specific antibodies to be detected by immunological based testing. For HCR, studies indicate that the window period may be reduced by 40-60 days from the total window period with antibody testing of 70 days. For HIV, studies suggest that the window period is reduced by 6 days (from a 22 day window period to a 16 days window period) with the use of the HIV-1 p24 antigen test, with a further reduction in time for NAT based testing. A very small percentage of donors, about one per 4 million for HIV-1 and one per 275,000 for HCV, donate during the window period and test negative for these viruses based on conventional immunological testing procedures. Nucleic acid testing (e.g. with the INVADER assay) would allow the window period to be reduced, thus preventing the spread of infectious disease through the blood supply.

Recently, nucleic acid testing on pooled blood samples (e.g. mini-pools) has also been initiated by a number of blood banks across the United States (See, Gallarda et al., Molecular Diagnostics, 5(1):11-22, 2000 herein incorporated by reference). Pooling blood samples for low prevalence disease is good way to reduce the cost of testing individual blood samples (or other biological fluid samples) as most of the samples will be negative. One reason pooling is gaining acceptance is the fact that in Africa, nearly 30 percent of all blood donors go untested in a given month because there is not enough money to procure test kits for everyone. This is particularly devastating give the dramatic rise of AIDS in Africa. Pooling allows many samples to be tested (and cleared) with one test. Pooled batches that are found to be contaminated can then allow the individual samples making up the pool to be identified as requiring further screening.

The current size if the pools (called “mini-pools” because only a small sample from each individual sample is mixed into a test pool) being tested in the United States by NAT is 16 and 24 individual samples (See, Stramer, Curr Opin Hematol, Nov;7(6):387-91, 2000, herein incorporated by reference in its entirety). Examples of vendors supplying reagents for testing are Gen-Probe (transcription-mediated amplification (TMA) technology, distributed by Chiron Corp.), and Roche Molecular Systems (polymerase chain reaction, PCR, technology). Both of these methods rely on amplified the target nucleic acid sequences prior to detection in the pooled sample. The tests for both groups have been qualified to detect six described HCV genotypes, as well as described HIV-1 subtypes (See, Stramer above). Sensitivity levels claimed to be achieved by these technologies are about the same, with a reported sensitivity of about 100 genome copies per mL for HCV and close to 50 copies per mL for HIV-1 (See, Stramer). Importantly, the methods of the present invention, in some embodiments, allow for an increased sensitivity level without requiring prior amplification of the target nucleic acid sequence.

Pooling samples of blood or other biological fluids causes a dilution effect. Therefore, statistical methods may be employed to determine the appropriate number of samples that can be pooled (e.g. in a min-pool) for safe and effective testing. For example, the estimated prevelence of the target nucleic acid sequence in a population, and the sensitivity of the detection assay may be employed to determined. Examples of such methods and statistical methods for determining appropriate pooled samples sizes are found in U.S. Pat. No. 6,063,563 and Hammick et al., Internal. Statist. Rev. 62, 319-31, 1994, both of which are incorporated by reference. Also examples of devices useful for forming pools (e.g mini-pools) for testing biological fluids such as blood are found in U.S. Pat. No. 6,063,563, hereby incorporated by reference.

III. Detection of RNA Targets by INVADER-Directed Cleavage

In addition to the clinical need to detect specific DNA sequences for infectious and genetic diseases in pooled samples (e.g. blood samples), there is a need for technologies that can quantitatively detect target nucleic acids in pooled samples that are composed of RNA. For example, a number of viral agents, such as hepatitis C virus (HCV) and human immunodeficiency virus (HIV) have RNA genomic material, the quantitative detection of which can be used as a measure of viral load or presence in a blood samples or pooled blood sample (or other biological fluid).

Hepatitis C virus (HCV) infection is the predominant cause of post-transfusion non-A, non-B (NANB) hepatitis around the world. In addition, HCV is the major etiologic agent of hepatocellular carcinoma (HCC) and chronic liver disease world wide. HCV contamination of blood and the blood supply is of great concern in the United States and around the world. The genome of HCV is a small (9.4 kb) RNA molecule. In studies of transmission of HCV by blood transfusion it has been found the presence of HCV antibody, as measured in standard immunological tests, does not always correlate with the infectivity of the sample, while the presence of HCV RNA in a blood sample strongly correlates with infectivity. Conversely, serological tests may remain negative in immunosuppressed infected individuals, while HCV RNA may be easily detected (Cuthbert, Clin. Microbiol. Rev., 7:505 [1994]).

The need for and the value of developing a probe-based assay for the detection the HCV RNA blood samples is clear. The polymerase chain reaction has been used to detect HCV in clinical samples, but the problems associated with carry-over contamination of samples has been a concern. Direct detection of the viral RNA without the need to perform either reverse transcription or amplification (e.g. with the INVADER assay) allows the elimination of several of the points at which existing assays may fail.

The genome of the positive-stranded RNA hepatitis C virus comprises several regions including 5′ and 3′ noncoding regions (i.e., 5′ and 3′ untranslated regions) and a polyprotein coding region that encodes the core protein (C), two envelope glycoproteins (E1 and E2/NS1) and six nonstructural glycoproteins (NS2-NS5b). Molecular biological analysis of the HCV genome has showed that some regions of the genome are very highly conserved between isolates, while other regions are fairly rapidly changeable. The 5′ noncoding region (NCR) is the most highly conserved region in the HCV. These analyses have allowed these viruses to be divided into six basic genotype groups, and then further classified into over a dozen sub-types (the nomenclature and division of HCV genotypes is evolving; see Altamirano et al., J. Infect. Dis., 171:1034 (1995) for a recent classification scheme). The present invention provides methods for detecting HCV and other viral target sequences in pooled samples, such as blood samples. The following U.S. patents contain descriptions of exemplary HCV sequences that may be detected by the methods of the present invention: U.S. Pat. Nos. 6,346,375; 6,297,003; 6,210,962; 6,153,421; 6,150,087; 6,127,116; 6,110,465; 6,096,498; 6,074,816; 6,054,264; 6,027,729; 6,020,122; 6,001,990; 5,919,454; 5,879,904; 5,874,565; 5,871,962; 5,866,139; 5,863,719; 5,830,635; 5,763,159; 5,750,331; 5,747,241; 5,714,596; 5,712,088;, 5,645,983; 5,625,034; 5,610,009; 5,580,718; 5,576,302; 5,527,669; 5,514,539; 6,297,370; 6,217,872; 6,214,583; 6,190,864; 6,171,784; 6,071,693; 6,051,696; 5,998,130; 5,959,092; 5,910,405; 5,863,719; 5,847,101; 5,871,903; 5,851,759; 5,846,704; 5,837,442; 5,747,239; 5,550,016; 5,427,909; 6,379,886; 6,153,421; 5,914,228; and 5,372,928, all of which are hereby incorporated by reference. The following European patents also contain descriptions of exemplary HCV sequences that may be detected by the methods of the present invention: EP0775216; EP0637342; EP0318216; EP0398748; and EP0543924; all of which are hereby incorporated by reference.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); DS (dextran sulfate); and ° C. (degrees Centigrade).

### EXAMPLE 1

### APOC4 Detection by the INVADER Assay in Pooled Samples

This example describes the detection of a polymorphism in the APOC4 gene. In particular, this example describes the use of the INVADER assay to detect a mutation in the APOC4 gene in pooled samples.

In this example, genomic DNAs were isolated from blood samples from several individual donors, and were characterized by invasive cleavage for the T/C polymorphism in codon 96 of the APOC4 gene (See, Allan, et al., Genomics Jul. 20, 1995;28(2):291-300, hereby incorporated by reference). The APOC4 assay used 5′ GATTCGAGGAACCAGGCCTTGGTGT (SEQ ID NO:1) 3′ as the invasive oligonucleotide and either 5′ ATGACGTGGCAGACAGCGGACCCAGGTCC-PO₄3′ (SEQ ID NO:2) or 5′ ATGACGTGGCAGACCGCGGACCCAGGTCC-PO₄3′ (SEQ ID NO:3) as primary signal probes for the T (Leu96) and the C (Pro96) alleles, respectively. The secondary target and probe were 5′ CGGAGGAAGCGTTAGTCTGCCACGTCAT-NH₂ 3′ (SEQ ID NO:4) and 5′ FAM-TAAC[Cy3]GCTTCCTGCCG 3′, respectively (SEQ ID NO:5).

All oligonucleotides were synthesized using standard phosphoramidite chemistries. Primary probe oligonucleotides were unlabeled. The FRET probes were labeled by the incorporation of Cy3 phosphoramidite and fluorescein phosphoramidite (Glen Research, Sterling, Va.). While designed for 5′ terminal use, the Cy3 phosphoramidite has an additional monomethoxy trityl (MMT) group on the dye that can be removed to allow further synthetic chain extension, resulting in an internal label with the dye bridging a gap in the sugar-phosphate backbone of the oligonucleotide. Amine or phosphate modifications, as indicated, were used on the 3′ ends of the primary probes and the secondary target oligonucleotides to prevent their use as invasive oligonucleotides. 2′-O-methyl bases in the secondary target oligonucleotides are indicated by underlining and were also used to minimize enzyme recognition of 3′ ends. Approximate probe melting temperatures (Tₘs) were calculated using the Oligo 5.0 software (National Biosciences, Plymouth, Minn.; non-complementary regions were excluded from the calculations.

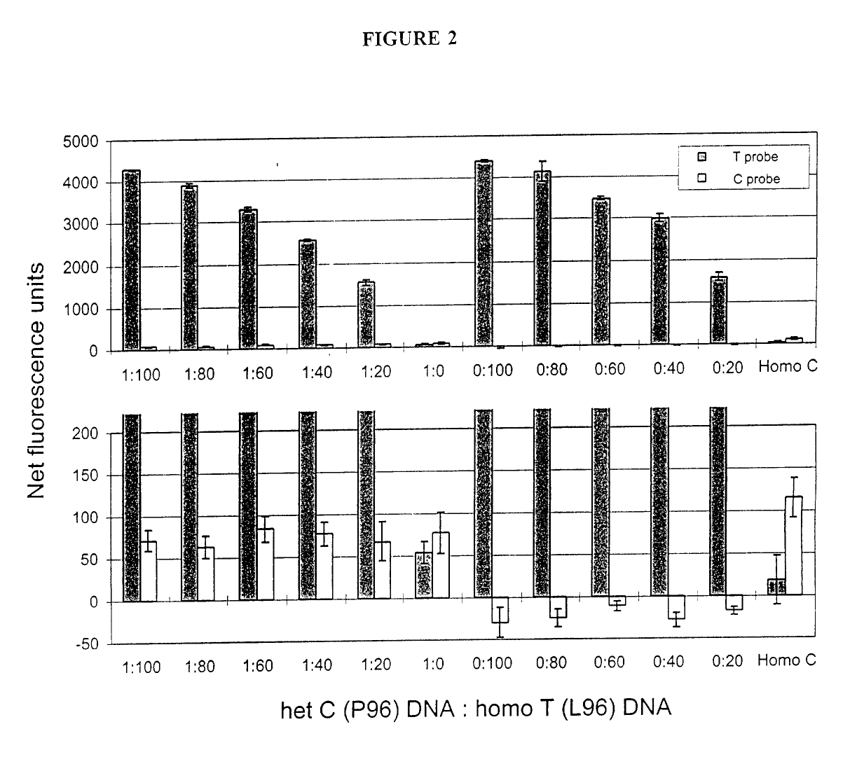

Pooled samples were constructed by diluting the heterozygous (het) DNA into DNA that is homozygous T (L96) at this locus. The test reactions contained 0.08 to 8 μg of T (L96) genomic DNA per reaction, and the het DNA was held at 0.08 μg, thus creating a set of mixtures in which het DNA represented from 50% down to 1% of the total DNA in the sample (See, FIG. 2). The actual representation of the C (P96) allele ranged from 25% down to 0.5% of the copies of this gene in the mixed samples. Controls included reactions having either all T (L96) DNA at each of the various DNA levels, or all het DNA at the 80 ng level. In addition, a sample of DNA that is homozygous for the C (P96) allele was tested (FIG. 2).

For all the INVADER assay reactions, 4 pmol of invasive probe, 40 pmol of FRET probe, and 20 pmol of secondary target oligonucleotide were combined with genomic DNA in 34 μl of 10 mM MOPS (pH 7.5) with 1.6% PEG. Reactions with the C (Pro96) allele of the APOC4 gene contained 80 ng of DNA heterozygous for this allele, and included DNA homozygous for the T (Leu96) allele at the indicated ratios. Samples were overlaid with 15 μl of Chill-Out liquid wax and heated to 95° C. for 5 min to denature the DNA. Upon cooling to 67° C. the reactions were started by the addition of 400 ng of Cleavase VIII enzyme, 15 pmol of either the T (Leu96) or the C (Pro96) primary signal probe, and MgCl₂ to a final concentration of 7.5 mM. The plates were incubated for 2 hours at 67° C., cooled to 54° C. to initiate the secondary (FRET) reaction, and incubated for another 2 hours. The reactions were then stopped by addition of 60 μl of TE. The fluorescence signals were measured on a Cytofluor fluorescence plate reader at excitation 485/20, emission 530/25, gain 65, temperature 25° C. Three replicates were done for each reaction and for no-target controls. The average signal for each target DNA was calculated, the average background from the no-target controls was subtracted, and the data plotted using Microsoft Excel.

The results of this example are shown in FIG. 2. As shown in this figure, the C (P96) allele was easily detected in all reactions, including that in which it was present in only 0.5% of the APOC4 alleles present in the mixture. These data indicate that the invasive cleavage reactions can be used for population analysis using pooled DNA samples. This has the double advantage of reducing the number of assays required to verify a new SNP, and of allowing the use of one large preparation of pooled DNA for numerous tests, thereby reducing the influence of sample-to-sample variations in DNA purity.

The above example demonstrates that the INVADER assay may be used to screen a population. A sample of mixed DNA to be analyzed should be large enough to bring the low-frequency alleles into the detectable range, e.g., 80 to 100 ng of the variant genome in these 40 μl reactions. As shown above in this Example, a sample of 8 to 10 μg of mixed DNA allowed detection of alleles present at 0.5 to 1% of the population under these conditions. In addition, the DNA from any one individual ideally should not be present in a large enough quantity to generate a detectable signal when an aliquot of the pool is tested. Creating a pool of several hundred individuals should guarantee that any detected signal reflects a contribution from many individuals in the pool. Finally, the use of a second probe set as an internal standard would allow the signals to be normalized from reaction to reaction, and would allow the prevalence of any SNP to be measured more accurately.

### EXAMPLE 2

### CFTR Detection by the INVADER Assay in Pooled Samples

This example describes the detection of a polymorphism in the CFTR gene. In particular, this example describes the use of the INVADER assay to detect the ΔF508 mutation in the CFTR gene in a pooled sample.

For INVADER assay analysis of the ΔF508 mutation, the primary probe set comprised 5′ ATATTCATAGGAAACACCAAG 3′ (SEQ ID NO:6) as the invasive oligonucleotide and either 5′ AACGAGGCGCACAGATGATATTTTCTTTAA 3′ (SEQ ID NO:7) or 5′ ATCGTCCGCCTCTGATATTTTCTTTAATGG 3′ (SEQ ID NO:8) as signal probes for the wild type and the mutant alleles. The secondary reaction components were designed to function optimally at a temperature at least 5 degrees below the primary reaction temperature.

All oligonucleotides described were synthesized using standard phosphoramidite chemistries. Primary probe oligonucleotides were unlabeled. The FRET probes were labeled by the incorporation of Cy3 phosphoramidite and fluorescein phosphoramidite (Glen Research, Sterling, Va.). While designed for 5′ terminal use, the Cy3 phosphoramidite has an additional monomethoxy trityl (MMT) group on the dye that can be removed to allow further synthetic chain extension, resulting in an internal label with the dye bridging a gap in the sugar-phosphate backbone of the oligonucleotide. One nucleotide was omitted at this position to accommodate the dye. Amine modifications were used on the 3′ ends of the primary probes, the secondary target and the arrestor oligonucleotides to prevent their use as invasive oligonucleotides. 2′-O-methyl bases are indicated by underlining and are also used to minimize enzyme recognition of 3′ ends. Approximate probe melting temperatures were calculated using the Oligo 5.0 software (National Biosciences, Plymouth, Minn.); noncomplementary regions were excluded from the calculations.

DNA samples characterized for CFTR genotype were purchased from Coriell Institute for Medical Research (Camden, N.J.), catalog numbers NA07469 (heterozygous in the CFTR gene for both ΔF508 and R553X mutations) and NA01531 (homozygous ΔF508). To determine what dose of a mutant could be detected within a pooled sample using the FRET-sequential invasive cleavage approach, DNA that is the heterozygous for the ΔF508 mutation in the CFTR gene was diluted into DNA that is homozygous wild type at that locus. The test reactions contained 0.1 to 2.6 μg of the total genomic DNA per reaction, and the mutant DNA was held at 0.1 μg, thus creating a set of mixtures in which mutant DNA represented from 50% down to 4% of the total DNA in the sample. Because the mutant DNA was heterozygous at the 508 locus, the actual allelic representation ranged from 25% down to 2% of the DNA in the mixed samples. Controls included reactions having either all wt at each of the various DNA levels, or all heterozygous mutant DNA at the 100 ng level. In addition, a sample of DNA that is homozygous for the ΔF508 mutation was tested.

DNA concentrations were estimated using the PicoGreen method. 4 pmol of INVADER probe, 40 pmol of FRET probe, and 20 pmole of secondary target oligonucleotide were combined with genomic DNA in 34 μl of 10 mM MOPS (pH 7.5) with 4% PEG. Samples were overlaid with 15 μl of Chill-Out liquid wax and heated to 95° C. for 5 min to denature the DNA. Upon cooling to 62° C. the reactions were started by the addition of 400 ng of AfuFEN1 enzyme, 15 pmole of either wt or mutant primary probe, and MgCl₂ to a final concentration of 7.5 mM. The plates were incubated for 2 hours at 62° C., cooled to 54° C. to initiate the secondary (FRET) reaction, and incubated for another 2 hours. The reactions were then stopped by addition of 60 μl of TE. The fluorescence signals were measured on a Cytofluor fluorescence plate reader excitation 485/20, emission 530/25, gain 65, temperature 25° C. Three replicates were done for each reaction and for no-target controls. The average signal for each target DNA was calculated, the average background from the no-target controls was subtracted, and the data plotted using Microsoft Excel.

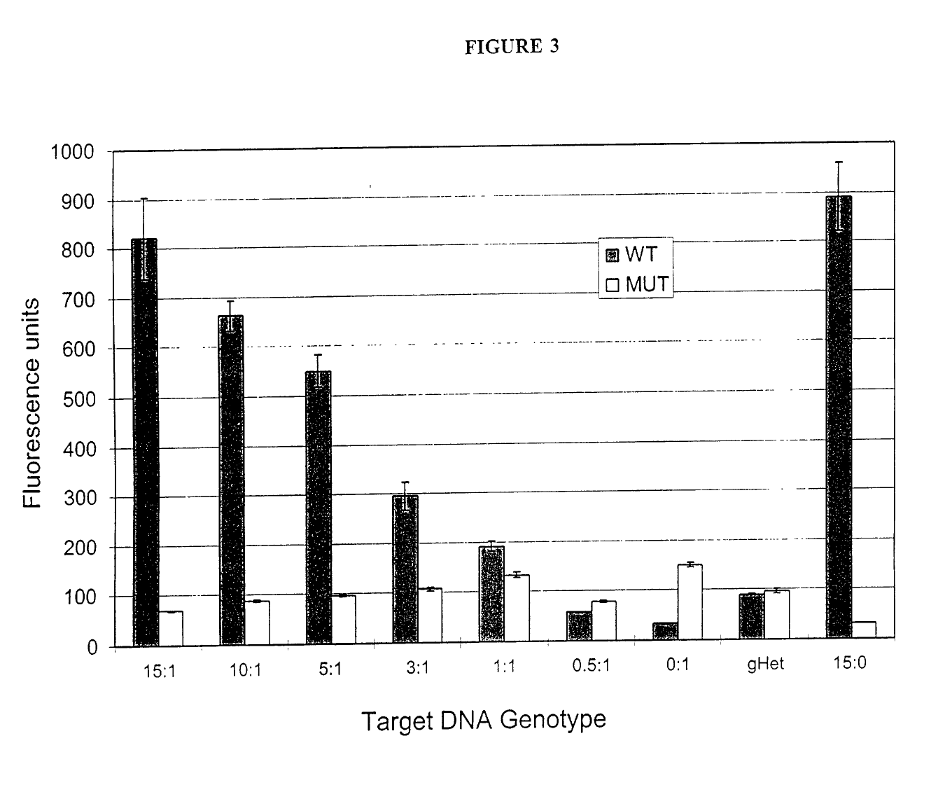

The results of this Example are presented in FIG. 3. Analysis of the signal from the mutant allele shows that it is not noticeably inhibited by substantial increases in the amount of wild type DNA, and the ΔF508 mutant DNA could be easily detected when present as only 2% of the mixture (FIG. 3). These data indicate that the invasive cleavage reactions can be used for population analysis using pooled DNA samples. This has the double benefit of reducing the number of assays required to verify a new SNP, and of allowing the use of one large, preparation of the pooled DNA to be used for numerous tests, thereby reducing the influence of sample-to-sample variations in DNA purity.

Application of the INVADER assay to screen populations is possible given the results presented in this example. In preferred embodiments for population screening, the DNA contribution from each individual should be equal, and the DNA from any one individual should not be present in a large enough quantity to generate a detectable signal when an aliquot of the pool is tested. For example, for this system creating a large enough pool that any one person contributes less than 1 ng (e.g., 0.5 ng) to each reaction should guarantee that any detected signal reflects a contribution from many individuals in the pool. For other detection systems, limiting the DNA from any one individual to an amount less than the detection limit of the system, for example ⅕ to 1/10 the detection limit, should produce the desired effect. The use of a second probe set as an internal standard, for example, would allow the signals to be normalized from reaction to reaction, and would allow the prevalence of any SNP to be measured more accurately.

### EXAMPLE 3

### SNP Consortium No. TSC 0006429 Detection by the INVADER Assay in Pooled Samples

This example describes the detection of the Consortium No. TSC 0006429 (SNP 1831) mutation in pooled samples. DNA from 15 individuals was purchased from the Coriell Cell Repository and each sample was tested to identify the genotype at the SNP Consortium No. TSC 0006429 (SNP 1831) locus. Each reaction contained 40 ng of DNA from each individual, 0.366 μM primary probe. 0.0366 μM Invader oligonucleotide, 0.183 μM FRET Probe and 100 ng CLEAVASE VIII enzyme in a buffer of 10 mM MOPS (pH 7.5) with 7.5 mM MgCl₂.

The probes used were as follows (5′ to 3′):

(SEQ ID NO:9)

Invader: CTTACTTGACCTTGGGCCCAGTTATTTAACCTTCTAGACCT

(SEQ ID NO:10)

Probe T: CGCGCCGAGGATCAGTTTCTTCATCTCTAAAATGGA

(SEQ ID NO:11)

Probe G: CGCGCCGAGGCTCAGTTTCTTCATCTCTAAAATGGA

(SEQ ID NO:12)

Synthetic Target T: TGTATCCATTTTAGAGATGAAGAAACTGAG

(SEQ ID NO:13)

GGTCTAGAAGGTTAAATAACTGGGCCCAAGGTCAAGTAAGGG

(SEQ ID NO:14)

Synthetic Target G: TGTATCCATTTTAGAGATGAAGAAACTGAT

(SEQ ID NO:15)

GGTCTAGAAGGTTAAATAACTGGGCCCAAGGTCAAGTAAGGG

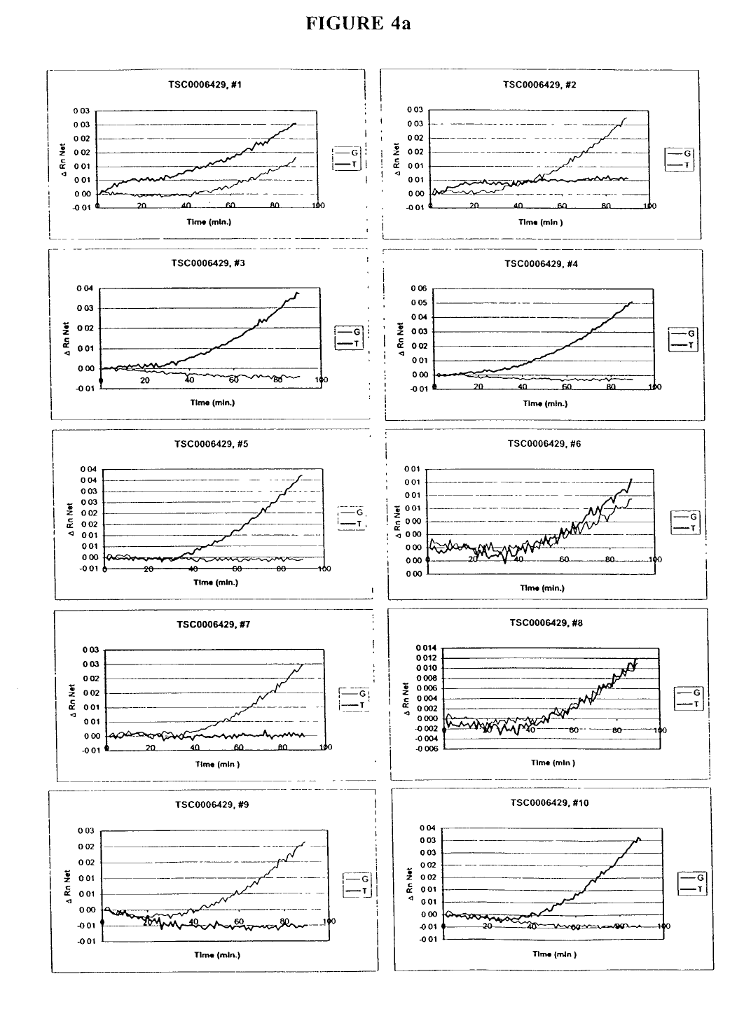

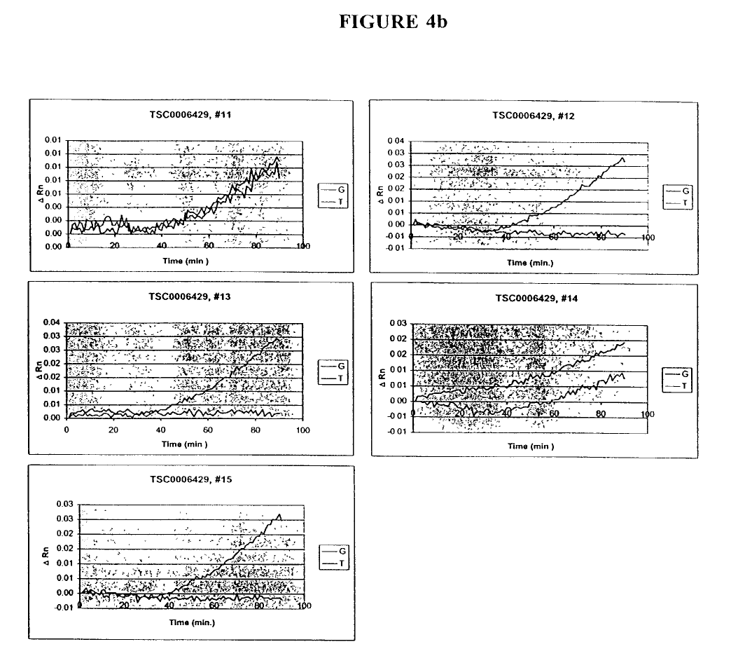

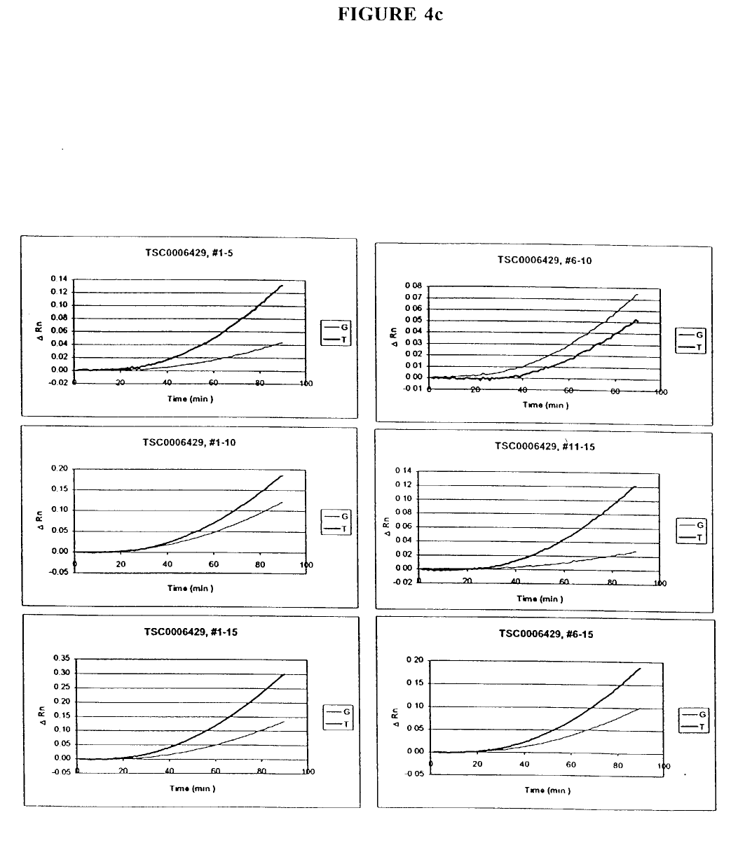

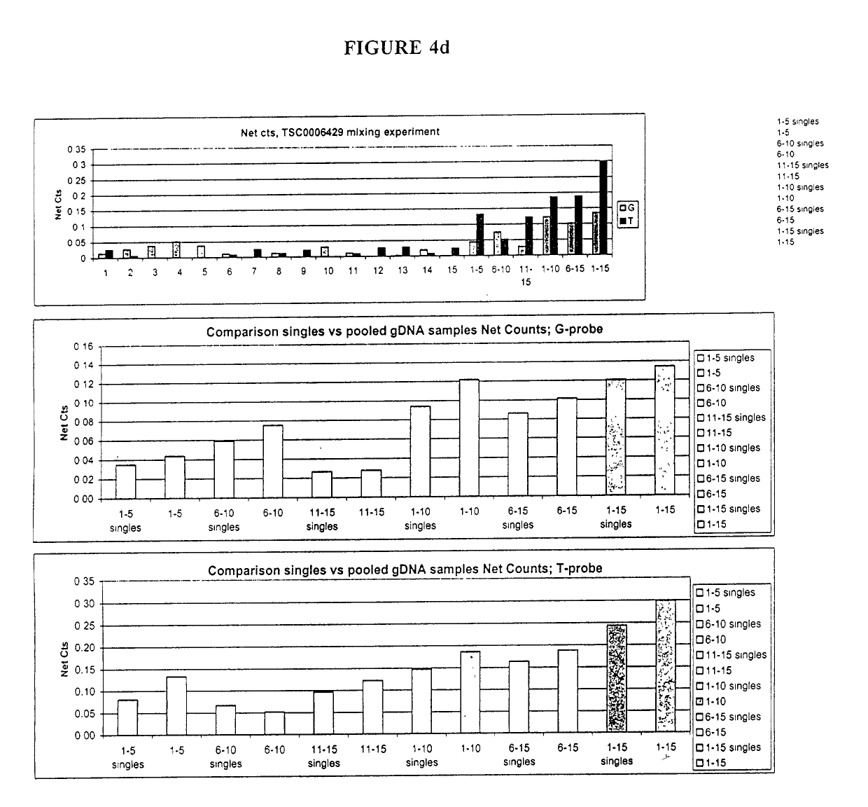

The assays were performed as described in Hall et al., PNAS, 97 (15):8272 (2000). Briefly, reaction were incubated at a constant temperature of 65° C. The data for each sample, produced using an ABI 7700 instrument for real-time reaction detection, are shown in the 15 panels of FIGS. 4a and 4b, with signals from the G allele shown as the light line and from the T allele shown as the dark line. The signal from each allele present in the mixture appears as an ascending curve reflecting the quadratic nature of the signal accumulation; the signal from any allele not present is essentially a straight line. These DNAs were then pooled in several combinations: Samples 1-5, 6-10, 11-15, 1-10, 6-15, and 1-15. The data panels are shown in FIG. 4c. FIG. 4d provides a comparison of the net fluorescence counts measured at the end of each reaction. From the results in 4a-b, the allele representation in each mixture can be calculated. Both FIGS. 4c and 4d demonstrate that the aggregate signals for each pool are proportional with respect to the final ratio of the alleles in the mix. The net fluorescence signals from the pooled samples are greater than those from the individuals because the amount of DNA from each person was held constant. For example, the assays run on DNA pooled from 5 individuals had 5 times as much DNA as the assays run on DNA from one individual.

As seen in this example, the real-time detection capabilities of the ABI 7700 can prove invaluable in detecting rare SNPs. Because the reaction is a two-step cascade, the real-time trace of signal accumulated in the Invader assay fits to a quadratic equation (i.e., the curves observed in FIGS. 4a-b and 4c), but background signal remains linear over the course of the reaction. Consequently, distinguishing signal arising from the genomic target from the background fluorescence is straightforward. This characteristic of the assay means that low-level signals from rare alleles can be resolved from background with more certainty.

### EXAMPLE 4

### Determination of Allele Frequencies by Comparison of Signals from Each Allele in Biplex INVADER Assays.

Measurement of different alleles within a single reaction removes concerns about sample-to-sample variations introducing inaccuracies into the measurements to be compared in the determination of allele frequency. Use of biplex (detection of two alleles or loci per reaction) or more complex multiplex (detection of more than two alleles or loci per reaction) configurations increases the through-put for allele frequency determination and facilitates comparisons of allele frequencies between different populations (e.g., affected vs. non-affected with a particular trait).

The following provides one example of a general protocol for the detection of two alleles in a DNA sample, and several examples wherein the protocol has been applied to the determination of alleles in samples. In this example, the signals are measured from fluorescein dye (FAM) and REDMOND RED dye (Red, Synthetic Genetics, San Diego, Calif.), each used on a separate FRET probe in combination with the Z28 ECLIPSE quencher (Synthetic Genetics, San Diego, Calif.). This protocol is provided to serve as an example and is not intended to limit the use of the methods or compositions of the present invention to any particular assay protocol or reaction configuration. Numerous fluorescent dyes and fluorophore/quencher combinations, and the methods of attaching and detecting such agents alone and in FRET combinations to nucleic acids are known in the art. Such other agents combinations are contemplated for use in the present invention and their use in these methods is within the scope of the present invention.

a. Procedure for Allele Frequency Determination in Pooled DNA

1. Determine the DNA concentration of each of the samples to be used in the INVADER Assay using the PICOGREEN reagents (procedure follows).

2. Mix the DNA samples at the desired ratios to mimic pools of genomic samples at specified allelic frequencies.

3. Denature the genomic DNA samples by incubating them at 95° C. for 10 min. Sample may then be placed on ice (optional).

4. Prepare a Probe/INVADER oligonucleotide/MgCl₂ mix by combining the 1.15 μL probe/INVADER oligonucleotide mix (3.5 μM of each primary probe and 0.35 μM INVADER oligonucleotide) and the 1.85 μL 24 mM MgCl₂ per reaction. Preparation of a master mix sufficient for testing of the complete set of samples is preferred.

5. Add 3 μl of the appropriate control or sample DNA target at 80 to 100 ng/μl (approximately 240-300 ng of genomic DNA) to the appropriate well of a 384-well biplex INVADER Assay FRET detection plate (Third Wave Technologies, Madison, Wis.). Each plate well contains 3 μl of a solution, dried after dispensing, containing 10 mM MOPS, 8% PEG, 4% glycerol, 0.06% NP 40, 0.06% Tween 20, 12 ug/ml BSA, 50 ng/ul BSA, 33.3 ng/ul CLEAVASE VIII enzyme, 1.17 μM FAM FRET probe (5′-FAM-TCT (Z28) AG CCG GTT TTC CGG CTG AGA GTC TGC CAC GTC AT-3′, SEQ ID NO:16) and 1.17 μM Red FRET Probe (5′-Red-TCT (Z28) TC GGC CTT TTG GCC GAG AGA CCT CGG CGC G-3′, SEQ ID NO:17).

6. Next, pipette 3 μl of Probe/INVADER oligonucleotide/MgCl₂ mix into the appropriate wells of the 384-well biplex INVADER Assay FRET detection plate.

7. Overlay each reaction with 6 μL of mineral oil.

8. Cover the plates with an adhesive cover and spin at 1,000 rpm in a Beckman GS-15R centrifuge (or equivalent) for 10 seconds to force the probe and target into the bottom of the wells.

9. Incubate the reactions at 63° C. for 3-4 hours in a thermal cycler or incubator such as a BioOven III. After 3-4 h incubation at 63° C., lower the temperature to 4° C. if a thermalcycler is being used or to RT if an incubator is being used.

10. Analyze the microtiter plate on a fluorescence plate reader using the following parameters:

| | | Wavelength/Bandwidth |
|---|---|---|
| FAM: | Excitation: | 485 nm/20 nm |
| | Emission: | 530 nm/25 nm |
| Red: | Excitation: | 560 nm/20 nm |
| | Emission: | 620 nm/40 nm |

b. Calculation of Fold-Over-Zero Minus 1 (FOZ-1):

The signals from each reaction are measured by comparison to the signal from a no-target control (the ‘zero’) and are expressed as a multiple of the signal from the ‘zero’ reaction. The factor one is subtracted to get the factor of actual signal over the background (e.g., for a sample having 1.5× the signal of the zero or 1.5 fold-over-zero, the amount of specific signal is 1.5-1, or 0.5).

Determine FOZ-1 as Follows:

FOZ-1 FAM Probe=((raw counts FAM probe 1, 485/530)/(raw counts from No Target Control FAM probe, 485/530))−1 .

FOZ-1 Red Probe=((raw counts Red probe 2, 560/620)/(raw counts from No Target Control Red probe, 560/620))−1

c. Calculation the Correction Factor (CF) as Follows

A correction factor can be calculated to accommodate any variations in the efficiencies of the cleavage reactions between the probe sets.

CF_{FAM}=(FOZ_{FAM}−1)/(FOZ_{Red}−1); CF_{Red}=(FOZ_{Red}−1)/(FOZ_{FAM}−1) of a heterozygous control.

$$For the FAM allelic frequency calculation : \frac{( {FOZ}_{FAM} - 1 ) / {CF}_{FAM} )}{( ( {FOZ}_{FAM} - 1 ) / {CF}_{FAM} ) + ( {FOZ}_{Red} - 1 )} \times 100 \\ For the Red allelic frequency calculation : \frac{( {FOZ}_{Red} - 1 ) / {CF}_{Red} )}{( ( {FOZ}_{Red} - 1 ) / {CF}_{Red} ) + ( {FOZ}_{FAM} - 1 )} \times 100$$

d. DNA Quantitation Procedure (Molecular Probes PICOGREEN Assay)

The PICOGREEN reagent is an asymmetrical cyanine dye (Molecular Probes, Eugene, Oreg.). Free dye does not fluoresce, but upon binding to dsDNA it exhibits a >1000-fold fluorescence enhancement. PICOGREEN is 10,000-fold more sensitive than UV absorbance methods, and highly selective for dsDNA over ssDNA and RNA.

1. Turn on the fluorescence plate reader at least 10 minutes before reading results. Use the following settings to read the PICOGREEN results:

| | Wavelength/Bandwidth |
|---|---|
| Excitation | ˜485 nm/20 nm |
| Emission: | ˜530 nm/25 nm |

2. Prepare 1× TE buffer (10 mM Tris-HCl, 1 mM EDTA, pH 7.5) from the 20× TE stock which is supplied in the PICOGREEN kit (to make 50 ml, add 2.5 ml of 20× TE to 47.5 ml sterile, distilled DNase-free water). 50 ml is sufficient for 250 assays.

3. Dilute DNA standards from 100 μg/ml to 2 μg/ml with 1× TE. For two standard curves, prepare 400 μl of a 2 μg/ml stock by adding 8 μl of the 100 μg/ml stock to 392 μl 1× TE.

4. Prepare the two standard curves in the microtiter plate as shown in the table:

| | Final | | Vol. (μl) |
|---|---|---|---|
| | [DNA] | Vol. (μl) 2 μg/ml | 1X TE |
| Plate Well | (ng/ml) | DNA Standard | Buffer |
| A1 & A2 | 0 | 0 | 100 |
| B1 & B2 | 25 | 2.5 | 97.5 |
| C1 & C2 | 50 | 5 | 95 |
| D1 & D2 | 100 | 10 | 90 |
| E1 & E2 | 200 | 20 | 80 |
| F1 & F2 | 300 | 30 | 70 |
| G1 & G2 | 400 | 40 | 60 |
| H1 & H2 | 500 | 50 | 50 |

5. For each unknown, add 2 μl of sample to 98 μl of 1× TE in the microplate well. Mix by pipetting up and down.

6. Prepare a 1:200 dilution of the PICOGREEN reagent in 1× TE. For each standard and each unknown sample, a volume of 100 μl is needed. For example, 2 standard curves with 8 points each will require 1.6 ml. To calculate the total volume of diluted PICOGREEN reagent needed, determine the total number of samples and unknowns will be tested and multiply this number by 100 μl (if using a multichannel pipet, make extra reagent). The PICOGREEN reagent is light sensitive and should be kept wrapped in foil while thawing and in the diluted state. Vortex well.

7. Add 100 μl of diluted PICOGREEN to every standard and sample. Mix by pipetting up and down.

8. Cover the microplate with foil and incubate at room temperature for 2-5 minutes.

9. Read the plate.

10. Generate a standard curve using the average values of the standards and determine the concentration of DNA in the unknown samples.

e. Measurement of Allele Frequencies in Genomic DNA Samples

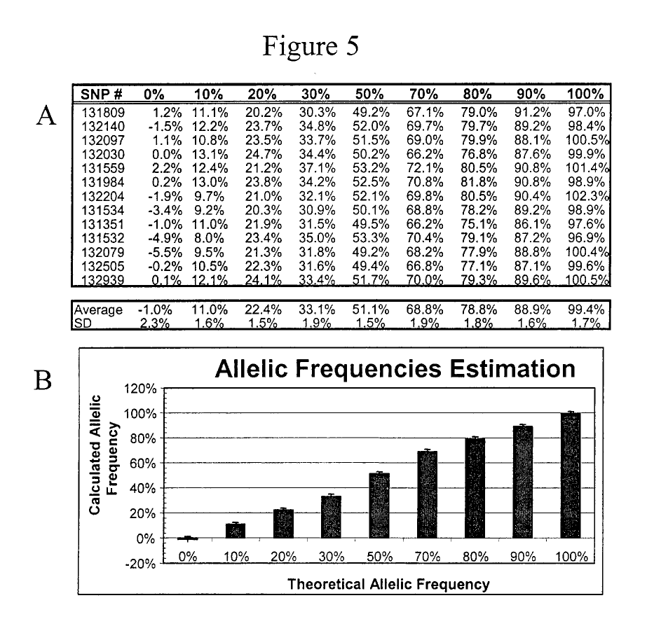

DNA samples having alleles at various frequencies were created by mixing different homozygous genomic DNA samples at different ratios. Each pool contained a total of 240 ng genomic DNA, and the reactions were carried out in 384-well plates as described above, at 63° C. for 3 hours. The measured signals are shown in FIG. 5A. The allelic frequencies were calculated based on the relative signal generated by the FAM and Red reporter dyes, and are displayed graphically in FIG. 5B. These data show the correlation between the theoretical or actual allelic frequency (the frequency intended to be created by mixing known amounts of DNA), compared to the allelic frequency calculated from the INVADER assay data.

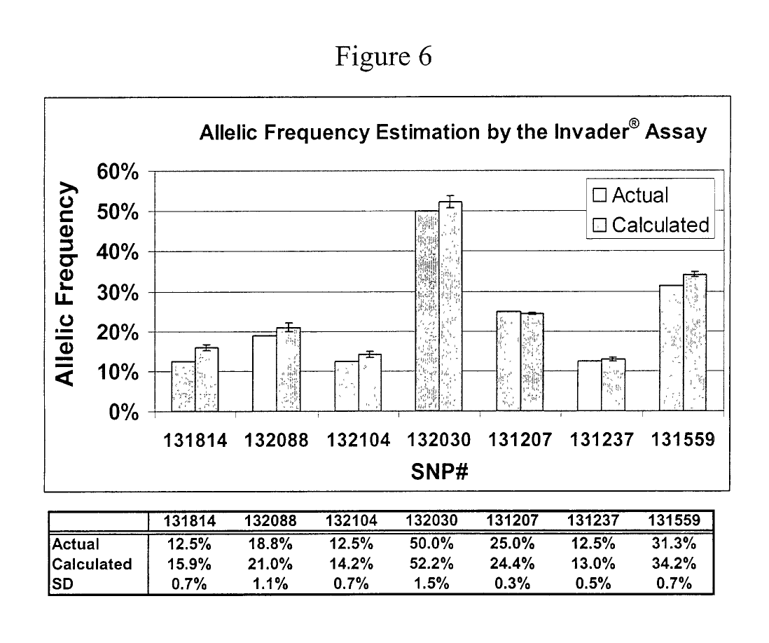

An 8-way pool of the genomic DNA of different individual was also tested. Each of the 8 DNA was previously characterized for each of 8 different SNP loci, so that the allelic frequency for each of the 8 SNPs in the pool was known. In this test, each pool contained a total of 300 ng genomic DNA, and the reactions were carried out in 384-well plates as described above, at 63° C. for 3 hours. The measured signals for the FAM channel, the rarer allele in each case, is shown in FIG. 6. The graph compares the known frequencies for each allele to the frequencies calculated from the INVADER assay data.

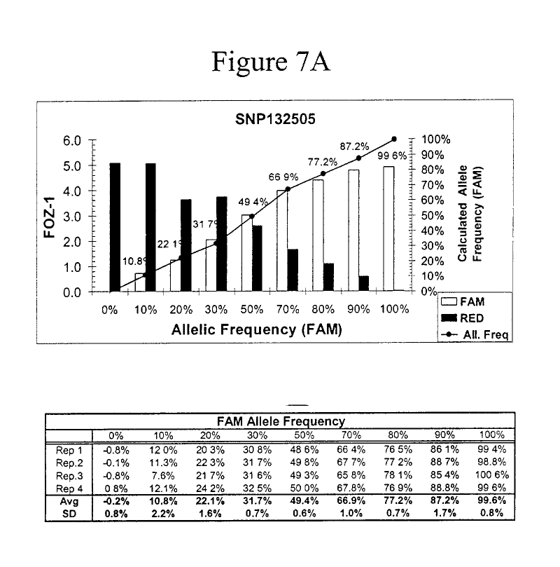

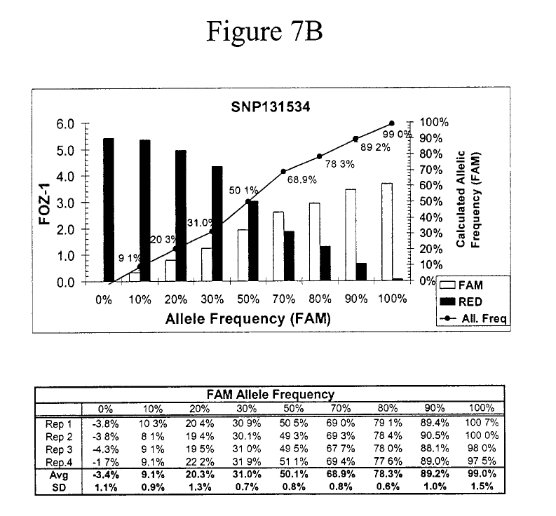

DNAs homozygous for each of two different SNPs (SNP132505 and SNP131534) were combined at various ratios to simulate genomic pools with different allelic frequencies. Each pool contained a total of 240 ng genomic DNA, and the reactions were carried out in 384-well plates as described above, at 63° C. for 3 hours. The allelic frequencies were calculated based on the relative signal generated by the FAM and Red reporter dyes, and are displayed graphically in FIGS. 7A and 7B.

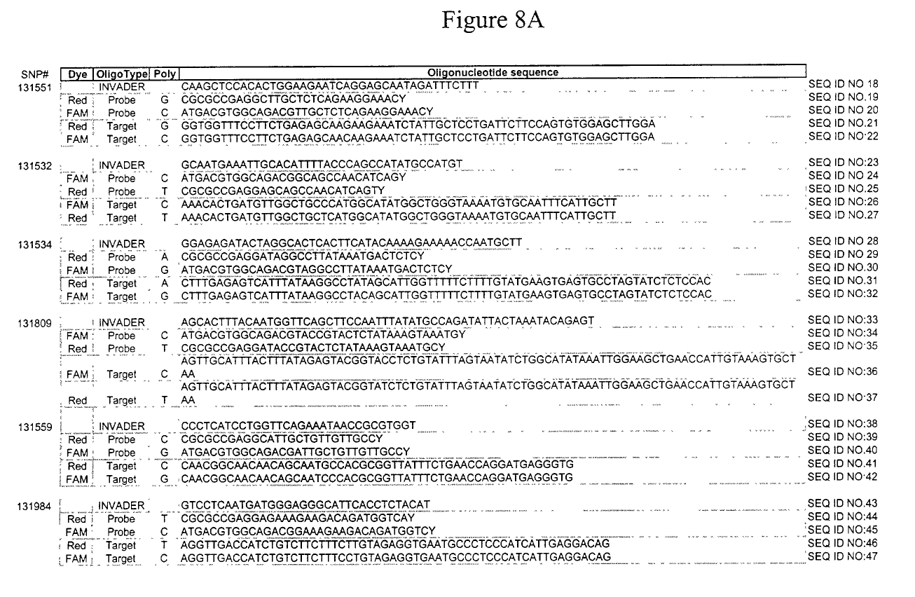

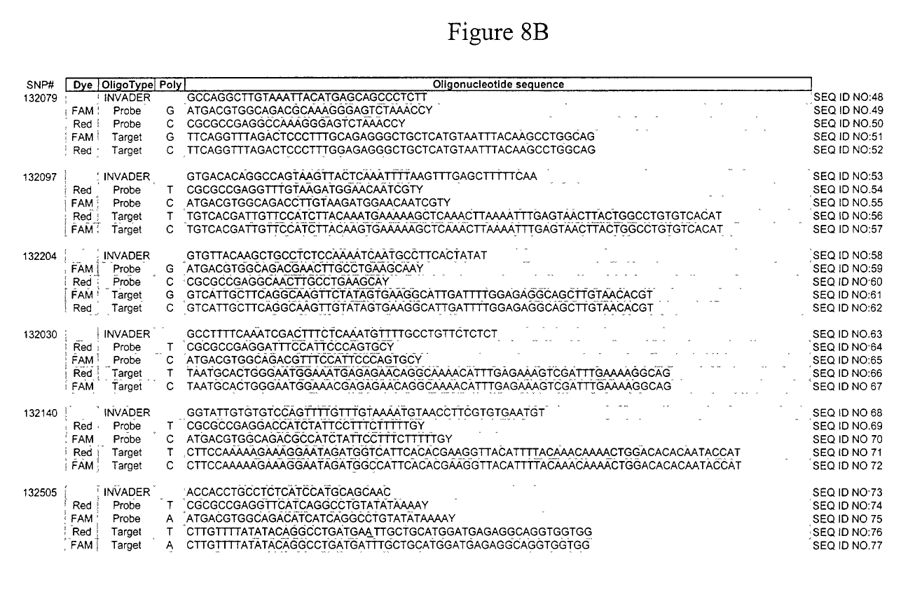

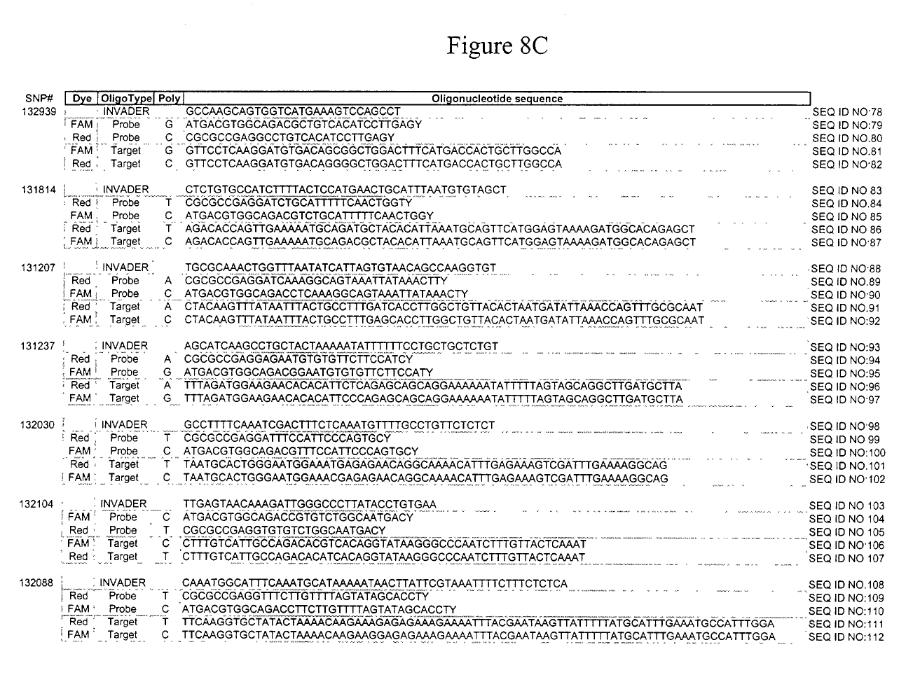

The probes used in the tests described above and additional probes sets suitable for use in the methods of the invention are shown in FIG. 8.

### EXAMPLE 5

### INVADER Assay Detection of HIV-1

The accessible sites method was employed in the design of probes for the INVADER assay-based detection of human immunodeficiency virus 1 (HIV-1) (WO0198537, incorporated herein by reference in its entirety)

Viral RNA was isolated from HIV-positive plasma samples using the QIAamp Viral RNA Kit (Qiagen) with the following protocol modifications. A dilution series was created by diluting purified HIV viral particles (strain IIIB, Advanced Biotechnologies, Inc.) in negative plasma (Lampire Biological Laboratories, Pipersville, Pa.). The plasma was certified to be negative for Hepatitis B surface antigen, HIV, Hepatitis C Virus, and syphilis. One ml of each plasma sample was first subjected to high-speed centrifugation at 23,500×g for 1 h at 4° C. to concentrate the virus, 930 μl of supernatant was removed and discarded. To lyse the particles, 280 μl of QIAgen buffer AVL were added and samples were incubated at 25° C. for 10 min. The lysate was applied to the spin column after the addition of 280 μl 100% ethanol, followed by one wash with 500 μl QIAgen AW2. 50 μl of heated distilled H₂O (70° C.) were added, columns were incubated at 70° C. for 5 min, and the eluted RNA was collected by centrifugation.

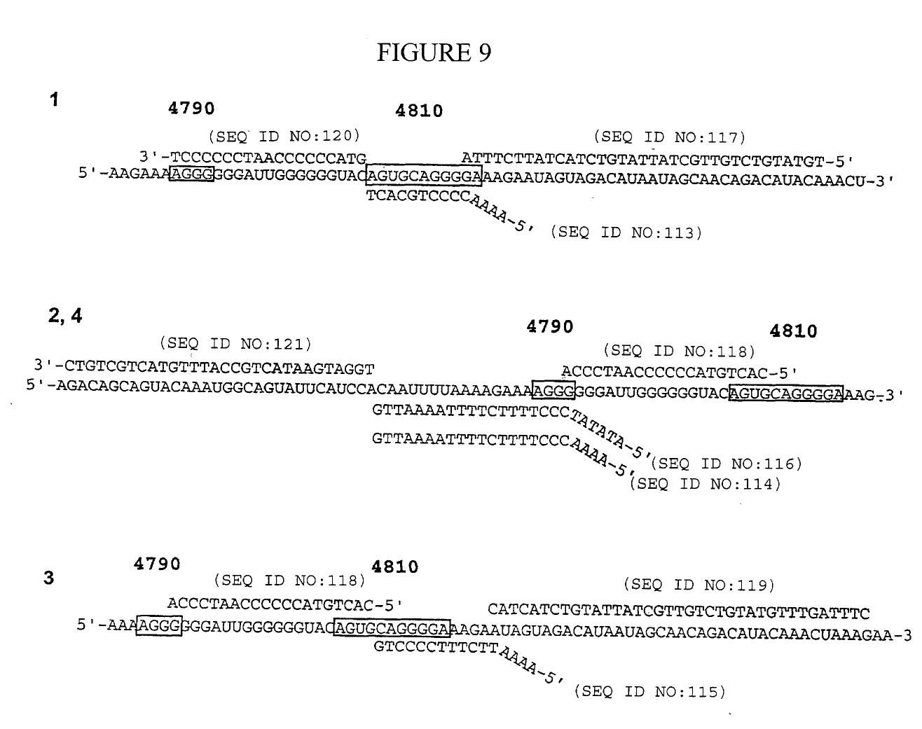

As seen in FIG. 9, for the pol site 4800, 4 different INVADER/probe oligonucleotide sets were tested (with and without stacking oligonucleotides). All of the designs position the probe oligonucleotide directly, in the accessible site. Designs 1, 2 and 4 position the probe cleavage site within the accessible site, while Design 3 positions the cleavage site just downstream of the accessible site, so that only the 3′ end of the probe is in the accessible site.

INVADER assays were performed in 10 μL total reaction volumes using 10 ng of CLEAVASE TTH DN (Third Wave Technologies; See PCT Publication WO 98/23774, herein incorporated by reference in its entirety), 1 nM of RNA, and 1 μM of 5′-labeled fluorescence probe and INVADER oligonucleotides in a final reaction buffer containing 500 ng/μL tRNA, 10 mM MOPS pH 7.5, 0.1 M KCl, and 5 mM MgCl₂. To determine the optimal reaction temperature for each probe/INVADER oligonucleotide set, temperature optimization were performed on a gradient thermocycler. Once the optimal temperature was determined, a one hour INVADER reaction was carried out, followed by cooling to 4° C. and addition of 2 μL of gel loading dye containing 90% formamide, and bromophenol blue in 10 mM Tris-HCl, 0.1 mM EDTA, pH 8. 5 μL of each reaction were then loaded on a 20% denaturing PAGE and allowed to run for 20 minutes and scanned as described above, using a 505 nm emission filter. Turnover rates were determined from the percentage of cleaved probe, as calculated from band intensities integrated using FMBIO-100 scanner software.

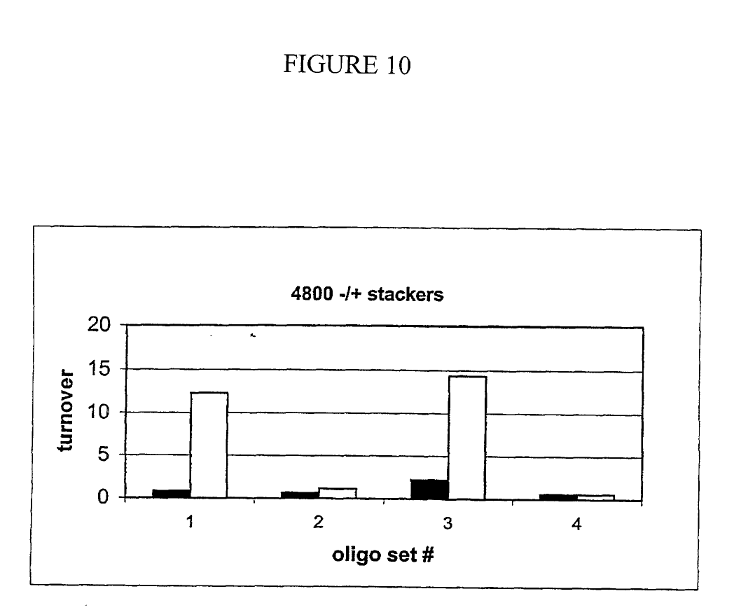

Reactions containing stacking oligonucleotides were performed as described in above, with the addition of 50 pmoles of a stacking oligonucleotide to the reaction. Results of the different designs and different reactions are represented graphically in FIG. 10. Design 3 used with stacking oligonucleotides gives the highest turnover rate, with the other 3 designs being comparable in performance. All four oligonucleotide sets performed better with the stacker than without, with the improvement being most dramatic in oligonucleotide sets 1 and 3. While not limiting the present invention to any particular mechanism, and while an understanding of these mechanisms is not necessary for the practice of the methods of the present invention, it is observed that the stacker oligonucleotides used for sets 1 and 3 are positioned to overlap or completely cover the adjacent accessible site, while the stackers for sets 2 and 4 cover sequence determined to be not accessible by the DP-RT method. Probe sets showing the greatest rate of signal accumulation (sets 1 and 3 from FIG. 9) were used to design a sequential INVADER assay (See e.g., U.S. Pat. No. 5,994,069 and PCT Publication WO 98/42873).

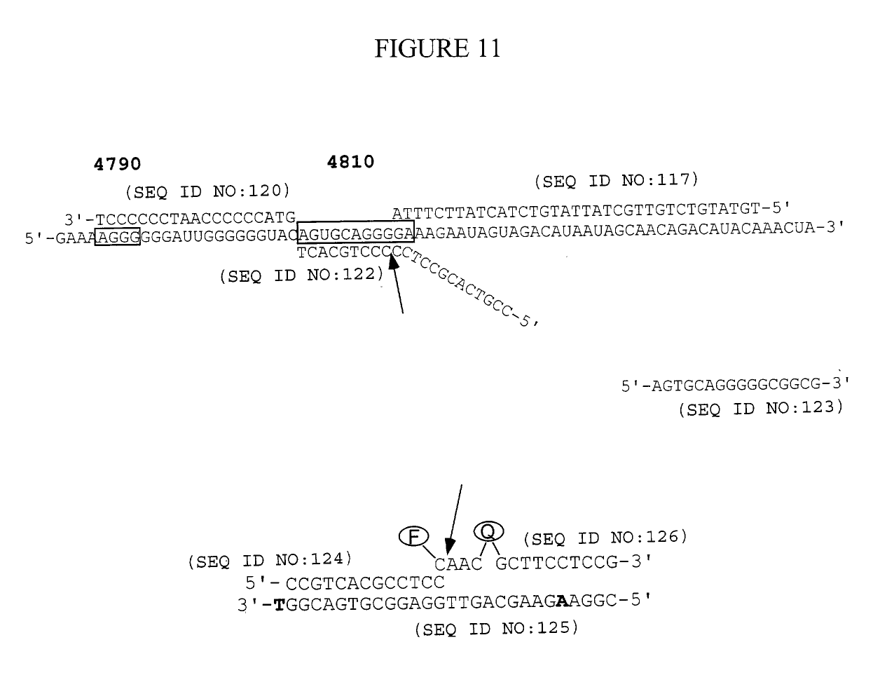

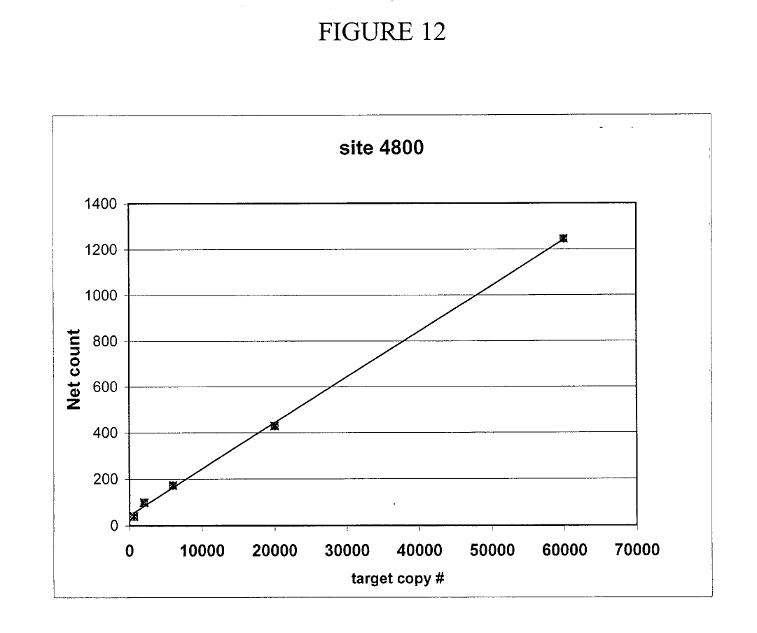

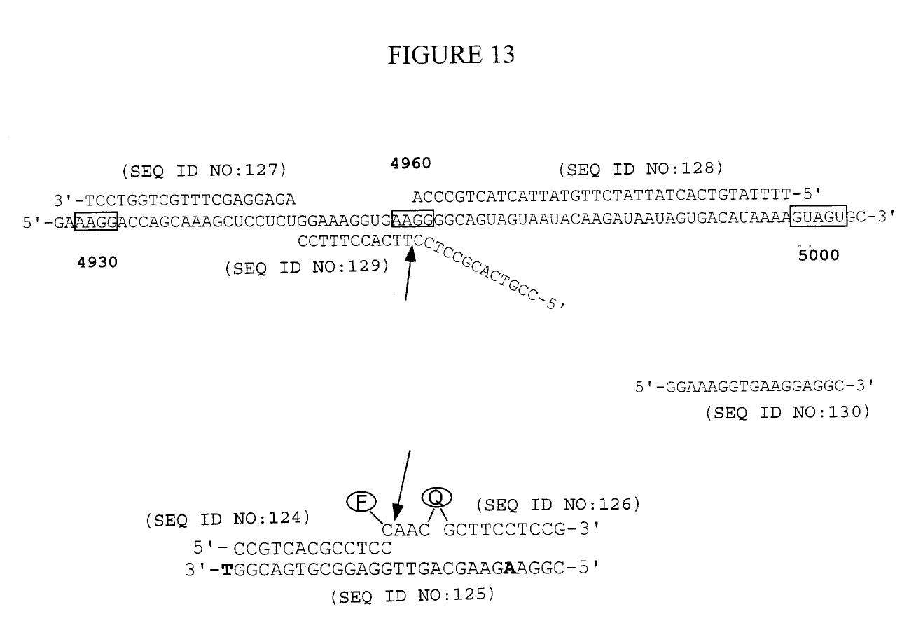

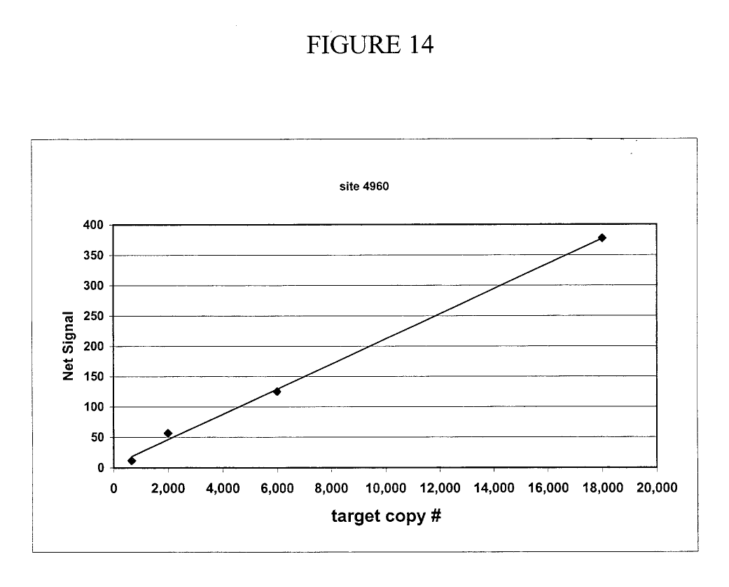

In testing different primary arms and secondary system sequences, set 3 proved problematic due to sequence similarity with the secondary systems and primary arms, resulting in aberrant hybridization. Set 1 was therefore used to detect HIV particles at a range of concentrations, with probe designs shown in FIG. 11. The viral samples were prepared as detailed above for the 1840 site, and the INVADER assay reactions were performed as described, with the resulting data shown in FIG. 12. Probe sets were also designed for the Pol and used to detect HIV-1 RNA at a range of concentrations. The probe set used is diagrammed in FIG. 13, with the results shown in FIG. 14.

### EXAMPLE 6

### INVADER Assay Detection of HCV

In order to develop a rapid and accurate method of detecting HCV present in infected individuals, the ability of the INVADER-directed cleavage reaction to detect HCV RNA was examined. Plasmids containing DNA derived from the conserved 5′-untranslated region of six different HCV RNA isolates were used to generate templates for in vitro transcription. The HCV sequences contained within these six plasmids represent genotypes 1 (four sub-types represented; 1a, 1b, 1c, and Δ1c), 2, and 3. The nomenclature of the HCV genotypes used herein is that of Simmonds et al. (as described in Altamirano et al., supra). The AIc subtype was used in the model detection reaction described below.

a) Generation of Plasmids Containing HCV Sequences

Six DNA fragments derived from HCV were generated by RT-PCR using RNA extracted from serum samples of blood donors; these PCR fragments were a gift of Dr. M. Altamirano (University of British Columbia, Vancouver). These PCR fragments represent HCV sequences derived from HCV genotypes 1a, 1b, 1c, Δ1c, 2c and 3a.

The RNA extraction, reverse transcription and PCR were performed using standard techniques (Altamirano et al., supra). Briefly, RNA was extracted from 100 μl of serum using guanidine isothiocyanate, sodium lauryl sarkosate and phenol-chloroform (Inchauspe et al., Hepatol., 14:595 [1991]). Reverse transcription was performed according to the manufacturer's instructions using a GeneAmp rTh reverse transcriptase RNA PCR kit (Perkin-Elmer) in the presence of an external antisense primer, HCV342. The sequence of the HCV342 primer is 5′-GGTTTTTCTTTGAGGTTTAG-3′ (SEQ ID NO:131). Following termination of the RT reaction, the sense primer HCV7 (5′-GCGACACTCCACCATAGAT-3′ [SEQ ID NO:132]) and magnesium were added and a first PCR was performed. Aliquots of the first PCR products were used in a second (nested) PCR in the presence of primers HCV46 (5′-CTGTCTTCACGCAGAAAGC-3′ [SEQ ID NO:133]) and HCV308 [5′-GCACGGT CTACGAGACCTC-3′ [SEQ ID NO:134]). The PCRs produced a 281 bp product that corresponds to a conserved 5′ noncoding region (NCR) region of HCV between positions −284 and −4 of the HCV genome (Altramirano et al., supra).

The six 281 bp PCR fragments were used directly for cloning or they were subjected to an additional amplification step using a 50 μl PCR comprising approximately 100 fmoles of DNA, the HCV46 and HCV308 primers at 0.1 μM, 100 μM of all four dNTPs and 2.5 units of Taq DNA polymerase in a buffer containing 10 mM Tris-HCl, pH 8.3, 50 mM KCl, 1.5 mM MgCl₂ and 0.1% Tween 20. The PCRs were cycled 25 times at 96° C. for 45 sec., 55° C. for 45 sec. and 72° C. for 1 min. Two microliters of either the original DNA samples or the reamplified PCR products were used for cloning in the linear pT7Blue T-vector (Novagen) according to manufacturer's protocol. After the PCR products were ligated to the pT7Blue T-vector, the ligation reaction mixture was used to transform competent JM109 cells (Promega). Clones containing the pT7Blue T-vector with an insert were selected by the presence of colonies having a white color on LB plates containing 40 μg/ml X-Gal, 40 μg/ml IPTG and 50 μg/ml ampicillin. Four colonies for each PCR sample were picked and grown overnight in 2 ml LB media containing 50 μg/ml carbenicillin. Plasmid DNA was isolated using the following alkaline miniprep protocol. Cells from 1.5 ml of the overnight culture were collected by centrifugation for 2 min. in a microcentrifuge (14K rpm), the supernatant was discarded and the cell pellet was resuspended in 50 μl TE buffer with 10 μg/ml RNAse A (Pharmacia). One hundred microliters of a solution containing 0.2 N NaOH, 1% SDS was added and the cells were lysed for 2 min. The lysate was gently mixed with 100 μl of 1.32 M potassium acetate, pH 4.8, and the mixture was centrifuged for 4 min. in a microcentrifuge (14K rpm); the pellet comprising cell debris was discarded. Plasmid DNA was precipitated from the supernatant with 200 μl ethanol and pelleted by centrifugation a microcentrifuge (14K rpm). The DNA pellet was air dried for 15 min. and was then redissolved in 50 μl TE buffer (10 mM Tris-HCl, pH 7.8, 1 mM EDTA).

b) Reamplification of HCV Clones to Add the Phage T7 Promoter for Subsequent in Vitro Transcription

To ensure that the RNA product of transcription had a discrete 3′ end it was necessary to create linear transcription templates that stopped at the end of the HCV sequence. These fragments were conveniently produced using the PCR to reamplify the segment of the plasmid containing the phage promoter sequence and the HCV insert. For these studies, the clone of HCV type Δ1c was reamplified using a primer that hybridizes to the T7 promoter sequence: 5′-TAATACGACTCACTATAGGG-3′ (SEQ ID NO:135; “the T7 promoter primer”) (Novagen) in combination with the 3′ terminal HCV-specific primer HCV308 (SEQ ID NO:134). For these reactions, 1 μl of plasmid DNA (approximately 10 to 100 ng) was reamplified in a 200 μl PCR using the T7 and HCV308 primers as described above with the exception that 30 cycles of amplification were employed. The resulting amplicon was 354 bp in length. After amplification the PCR mixture was transferred to a fresh 1.5 ml microcentrifuge tube, the mixture was brought to a final concentration of 2 M NH₄OAc, and the products were precipitated by the addition of one volume of 100% isopropanol. Following a 10 min. incubation at room temperature, the precipitates were collected by centrifugation, washed once with 80% ethanol and dried under vacuum. The collected material was dissolved in 100 μl nuclease-free distilled water (Promega).

Segments of RNA were produced from this amplicon by in vitro transcription using the RiboMAX™ Large Scale RNA Production System (Promega) in accordance with the manufacturer's instructions, using 5.3 μg of the amplicon described above in a 100 μl reaction. The transcription reaction was incubated for 3.75 hours, after which the DNA template was destroyed by the addition of 5-6 μl of RQ1 RNAse-free DNAse (1 unit/μl) according to the RiboMAX™ kit instructions. The reaction was extracted twice with phenol/chloroform/isoamyl alcohol (50:48:2) and the aqueous phase was transferred to a fresh microcentrifuge tube. The RNA was then collected by the addition of 10 μl of 3M NH₄OAc, pH 5.2 and 110 μl of 100% isopropanol. Following a 5 min. incubation at 4° C., the precipitate was collected by centrifugation, washed once with 80% ethanol and dried under vacuum. The sequence of the resulting RNA transcript (HCV1.1 transcript) is listed in SEQ ID NO:136.

c) Detection of the HCV1.1 Transcript in the INVADER-Directed Cleavage Assay

Detection of the HCV1.1 transcript was tested in the INVADER-directed cleavage assay using an HCV-specific probe oligonucleotide (5′-CCGGTCGTCCTGGCAAT XCC-3′ [SEQ ID NO:137]); X indicates the presence of a fluorescein dye on an abasic linker) and an HCV-specific INVADER oligonucleotide (5′-GTTTATCCAAGAAAGGAC CCGGTC-3′ [SEQ ID NO:138]) that causes a 6-nucleotide invasive cleavage of the probe.

Each 10 μl of reaction mixture comprised 5 pmole of the probe oligonucleotide (SEQ ID NO:137) and 10 pmole of the INVADER oligonucleotide (SEQ ID NO:138) in a buffer of 10 mM MOPS, pH 7.5 with 50 mM KCl, 4 mM MnCl₂, 0.05% each Tween-20 and Nonidet-P40 and 7.8 units RNasin® ribonuclease inhibitor (Promega). The cleavage agents employed were CLEAVASE A/G (used at 5.3 ng/10 μl reaction) or DNAPTth (used at 5 polymerase units/10 μl reaction). The amount of RNA target was varied as indicated below. When RNAse treatment is indicated, the target RNAs were pre-treated with 10 μg of RNase A (Sigma) at 37° C. for 30 min. to demonstrate that the detection was specific for the RNA in the reaction and not due to the presence of any residual DNA template from the transcription reaction. RNase-treated aliquots of the HCV RNA were used directly without intervening purification.

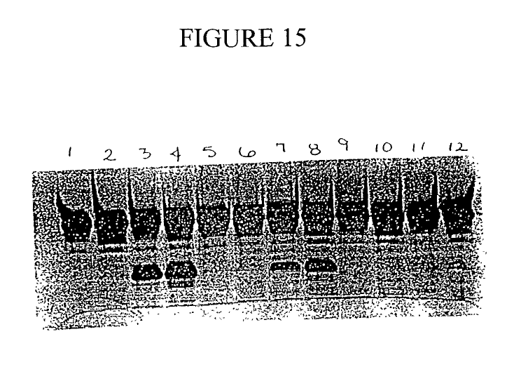

For each reaction, the target RNAs were suspended in the reaction solutions as described above, but lacking the cleavage agent and the MnCl₂ for a final volume of 10 μl, with the INVADER and probe at the concentrations listed above. The reactions were warmed to 46° C. and the reactions were started by the addition of a mixture of the appropriate enzyme with MnCl₂. After incubation for 30 min. at 46° C., the reactions were stopped by the addition of 8 μl of 95% formamide, 10 mM EDTA and 0.02% methyl violet (methyl violet loading buffer). Samples were then resolved by electrophoresis through a 15% denaturing polyacrylamide gel (19:1 cross-linked), containing 7 M urea, in a buffer of 45 mM Tris-Borate, pH 8.3, 1.4 mM EDTA. Following electrophoresis, the labeled reaction products were visualized using the FMBIO-100 Image Analyzer (Hitachi), with the resulting imager scan shown in FIG. 15.

In FIG. 15, the samples analyzed in lanes 1-4 contained 1 pmole of the RNA target, the reactions shown in lanes 5-8 contained 100 fmoles of the RNA target and the reactions shown in lanes 9-12 contained 10 fmoles of the RNA target. All odd-numbered lanes depict reactions performed using CLEAVASE A/G enzyme and all even-numbered lanes depict reactions performed using DNAPTth. The reactions analyzed in lanes 1, 2, 5, 6, 9 and 10 contained RNA that had been pre-digested with RNase A. These data demonstrate that the invasive cleavage reaction efficiently detects RNA targets and further, the absence of any specific cleavage signal in the RNase-treated samples confirms that the specific cleavage product seen in the other lanes is dependent upon the presence of input RNA.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, and molecular biology or related fields are intended to be within the scope of the following claims.

We claim:

1. A method of performing nucleic acid testing on a pooled sample, comprising:
a) providing;
i) a pooled sample, wherein said pooled sample comprises biological material combined from a plurality of individual samples; and
ii) INVADER assay reagents configured to detect the presence or absence of a target nucleic acid sequence; and
b) contacting said pooled sample with said INVADER assay reagents under conditions such that the presence or absence of said target nucleic acid sequence in said pooled sample is determined.

2. The method of claim 1, wherein said biological material comprises blood.

3. The method of claim 1, wherein said biological material comprises blood plasma.

4. The method of claim 1, wherein said target nucleic acid sequence is RNA.

5. The method of claim 1, wherein said target nucleic acid sequence is DNA.

6. The method of claim 1, wherein said target nucleic acid sequence is from a microorganism.

7. The method of claim 1, wherein said target nucleic acid sequence is from a virus.

8. The method of claim 1, wherein said target nucleic acid sequences is from a pathogen selected from HIV-1, HIV-2, HCV, HBV, HTLVI, HTLV2, and HCMV.

9. The method of claim 1, wherein said target nucleic acid comprises a first and second non-contiguous single-stranded regions separated by an intervening region comprising a double stranded regions, and wherein said INVADER assay reagents comprise; i) a bridging oligonucleotide capable of binding to said first and second non-contiguous single-stranded regions; ii) a second oligonucleotide capable of binding to a portion of said first non-contiguous single-stranded region; and iii) a cleavage means.

10. The method of claim 9, wherein said contacting causes either said second oligonucleotide or said bridging oligonucleotide to be cleaved.

11. The method of claim 1, wherein said plurality of individual samples comprises at least 5 individual samples.

12. The method of claim 1, wherein said plurality of individual samples comprises at least 16 individual samples.

13. The method of claim 1, further comprising, prior to step b), the step of performing polymerase chain reaction on said pooled sample such that said target nucleic acid sequence is amplified if present in said pooled sample.

14. The method of claim 1, wherein said contacting step is performed under conditions such that said target nucleic acid sequence is not amplified before said presence or said absence of said target nucleic acid sequence is determined.

15. A method of performing nucleic acid testing on a pooled sample, comprising:
a) providing;
i) a plurality of individual biological material samples; and
ii) INVADER assay reagents configured to detect the presence or absence of a target nucleic acid sequence; and
b) forming a sub-pool by combining a portion of each of said plurality of individual biological samples, and
c) contacting said sub-pool with said INVADER assay reagents under conditions such that the presence or absence of said target nucleic acid sequence in said sub-pool is determined.

16. The method of claim 15, wherein said contacting indicates that said target nucleic acid sequence is absent from said sub-pool.

17. The method of claim 16, further comprising the step of combining said plurality of individual biological samples into a primary pool.

18. The method of claim 15, wherein said contacting indicates that said target nucleic acid sequence is present in said sub-pool.

19. The method of claim 18, further comprising the step of screening each of said individual biological samples for the presence or absence of said target nucleic acid sequence.

20. A method of performing nucleic acid testing on a pooled sample, comprising:
a) providing;
i) a pooled sample, wherein said pooled sample comprises biological material combined from a plurality of individual samples; and
ii) INVADER assay reagents configured to detect measure the quantity of a target nucleic acid sequence present in a sample; and
b) contacting said pooled sample with said INVADER assay reagents under conditions such that the quantity of said target nucleic acid sequence present in said pooled sample is determined.

21. The method of claim 20, wherein said biological material comprises blood.

22. The method of claim 20, wherein said biological material comprises blood plasma.

23. The method of claim 20, wherein said target nucleic acid sequence is RNA.

24. The method of claim 20, wherein said target nucleic acid sequence is DNA.

25. The method of claim 20, wherein said target nucleic acid sequence is from a microorganism.

26. The method of claim 20, wherein said target nucleic acid sequence is from a virus.

27. A method for detecting an allele frequency of a polymorphism, comprising:
a) providing;
i) a pooled sample, wherein said pooled sample comprises target nucleic acid sequences from a plurality of individuals; and
ii) INVADER assay reagents configured to detect the presence or absence of a polymorphism; and
b) contacting said pooled sample with said INVADER assay reagents to generate a detectable signal; and
c) measuring said detectable signal, thereby determining a number of said target nucleic acid sequences that contain said polymorphism.

28. The method of claim 27, wherein said plurality comprises at least 10 individuals.

29. The method of claim 28, wherein said at least 10 individuals comprises at least 1000 individuals.

30. A method for detecting an allele frequency of a polymorphism, comprising:
a) providing;
i) a pooled sample, wherein said pooled sample comprises target nucleic acid sequences from a plurality of individuals; and
ii) INVADER assay reagents configured to generate distinct signals for each allele of a polymorphic locus in said target nucleic acid sequence;
b) contacting said pooled sample with said INVADER assay reagents to generate at least one distinct signal; and
c) measuring each of said at least one distinct signal, thereby determining a proportion of each allele of said polymorphic locus within said pooled sample.

31. The method of claim 30, wherein said measuring comprises detection of fluorescence.

32. The method of claim 30, wherein at least two distinct signals are generated in step b) and wherein said measuring comprises comparing said at least two distinct signals.

33. The method of claim 32, wherein said comparing comprises applying a correction factor to a measurement of at least one distinct signal.

34. A method for detecting a rare mutation comprising;
a) providing;
i) a sample from a single subject, wherein said sample comprises at least 10,000 target nucleic acid sequences,
ii) a detection assay capable of detecting a mutation in a population of target nucleic acid sequence that is present at an allele frequency of 1:1000 or less compared to wild type alleles; and
b) assaying said sample with said detection assay under conditions such that the presence or absence of a rare mutation is detected.

35. A method for detecting a rare mutation comprising;
a) providing;
i) a sample from a single subject, wherein said sample comprises at least 10,000 target nucleic acid sequences,
ii) a detection assay capable of detecting a mutation in a population of target nucleic acid sequence that is present at an allele frequency of 1:1000 or less compared to wild type alleles; and
b) assaying said sample with said detection assay under conditions such that an allele frequency in said sample of a rare mutation is determined.
